(19) United States
(12) Patent Application Publication
(10) Pub. No.: US 2015/0324544 A1
(43) Pub. Date: Nov. 12, 2015

(54) PAIN SURVEYING AND VISUALIZATION IN A HUMAN BODILY REGION

(71) Applicant: THE REGENTS OF THE UNIVERSITY OF MICHIGAN, Ann Arbor, MI (US)

(72) Inventors: Eric Maslowski, Ypsilanti, MI (US); Alexandre Dasilva, Ann Arbor, MI (US); Sean Petty, Canton, MI (US); Sean Sheehan, Ypsilanti, MI (US)

(21) Appl. No.: 14/707,172

(22) Filed: May 8, 2015

Related U.S. Application Data
(60) Provisional application No. 61/991,221, filed on May 9, 2014.

(51) Int. Cl. G06F 19/00; A61B 6/03; A61B 5/04; A61B 5/00

(52) U.S. Cl. CPC G06F 19/3437; A61B 5/0042; A61B 6/037; A61B 6/032; A61B 5/0075; A61B 5/04008; A61B 5/4824

(57) ABSTRACT

Techniques for pain surveying and visualization in a bodily region including a 3-dimensional rendering of a bodily region or an anatomical grid for presentation to a subject suffering from pain for collection of pain intensity and pain location information. A device is provided to the patients for display of the rendering of the bodily region or anatomical grid for collection of pain intensity and location information. A pain analysis module may then create an aggregate pain data set for visual data analyses, user reports, or data export focused on one or multiple region(s), as well as the entire body. The pain data sets may include patient data from a single patient or aggregated data from multiple patients.

### RELATED APPLICATION

This application claims the benefit under 35 U.S.C. §119(e) of U.S. Provisional Patent Application Ser. No. 61/991,221, entitled “PAIN SURVEYING AND VISUALIZATION IN A HUMAN BODILY REGION,” filed May 9, 2014, the entire disclosure of which is hereby expressly incorporated by reference herein.

### FIELD OF THE INVENTION

The present invention relates generally to measuring pain sensed by a subject or cohort, and, more particularly, to a device that collects sensed pain location, intensity, and subjective pain data from a subject with reference to a 3D model of a human bodily region.

### BACKGROUND OF THE INVENTION

A large number of people suffer from intense or chronic pain, particularly pain in the head, also known as cephalalgia. Intense or chronic pain is often debilitating and difficult for patients and physicians to manage. Most current treatment options are based on drugs, and often must be tested for effectiveness according to unsystematic trial-and-error techniques.

There are numerous reasons why it is so difficult for physicians and researchers to assess the effectiveness of pain treatments. Some conventional methods of pain assessment include the number scale (0-10 pain scale), the Wong-Baker FACES pain rating scale, the PQAS (Pain Quality Assessment Scale), VAS (Visual Analog Scale), VNRS (Verbal Numerical Rating Scale), VDS (Verbal Descriptor Scale), the BPI (Brief Pain inventory), and the Nurses Assessment, which are based on self-reporting by the patient. For neonates and infants, patients who cannot self-report pain, an observational test, the FLACC scale (Face, Legs, Activity, Cry, Consolability) may be used. Physiological data, such as a PET or MRI scan of the patient's brain during an episode of pain may also be used. Because pain is by definition what the patient senses, observational data and physiological data are limited. Pain self-reporting also has drawbacks because it is an inherently subjective procedure wherein two patients suffering from a similar level of pain may report disparate pain levels with reference to a numerical scale.

Another drawback of conventional pain assessments is that they often lack key data regarding the location of pain. For example, patients with trigeminal neuralgia may have varying degrees of pain in seemingly different regions. This data is lost when converted to a scalar or descriptive pain assessment rating. This loss of precision and accuracy increases the difficulty for the physician to prescribe a treatment dose appropriate for the level of pain. Moreover, these limitations make conventional pain assessments particularly poorly adapted to measure or track pain over time or to make treatment decisions based on pain location such as for treatments based on dermatomes, and overlapping pain conditions (e.g., fibromyalgia, temporomandibular disorders).

### SUMMARY OF THE INVENTION

The present disclosure relates to techniques for pain surveying and visualization in a bodily region. In some embodiments, the techniques of the present disclosure use a 3-dimensional rendering of a bodily region or an anatomical grid for presentation to a subject for collection of pain intensity and pain location information. A pain analysis module may then create an aggregate pain data set for visual data analyses, user reports, or data export focused on one or multiple region(s), as well as the entire body.

In one embodiment, the present disclosure is directed to a method of tracking and analyzing pain experienced by a subject. The method includes presenting, on a display, a visual rendering of a bodily region to track and analyze pain, where the visual rendering comprises a plurality of sub-regions collectively mapping the bodily region, where each sub-region is individually selectable by the subject. The method further includes receiving, from the subject interacting with the visual rendering on the display, identified pain data to create one or more pain heat maps, where each heat map comprises (i) a selection of one or more of the sub-regions and (ii) an indication of pain intensity for each of the selected one or more sub-regions, where the indication of pain intensity is a numeric value taken from a pain intensity scale. The method also includes developing, from the one or more pain heat maps, an aggregated pain data set for the bodily region, the aggregated pain data set including averaging data indicating an average pain intensity value over the one or more pain heat maps, sub-region coverage data indicating a percentage of plurality of sub-regions selected by the subject over the one or more pain heat maps, and summation data indicating a sum of total pain intensity from the one or more pain heat maps; and displaying a visual representation of the aggregated pain data set.

In another embodiment, the present disclosure is directed to an apparatus having a processor and a computer readable medium that includes instructions that when executed by the processor cause the apparatus to present, to a subject experiencing pain, a first visual rendering of a bodily region wherein the visual rendering comprises a plurality of sub-regions collectively mapping the bodily region; collect, from the subject experiencing pain, one or more pain data sets wherein each pain data set comprises pain intensity and pain location data corresponding to one or more of the plurality of sub-regions; develop, in a memory, the one or more pain data sets to produce an aggregate pain data set; and perform, in a pain analysis module, a data analysis of the aggregate pain data set to visualize the pain data for presentation on a second visual rendering of a bodily region.

While multiple embodiments are disclosed, still other embodiments of the present disclosure will become apparent to those skilled in the art from the following detailed description, which shows and describes illustrative embodiments of the disclosure. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

For a more complete understanding of the disclosure, reference should be made to the following detailed description and accompanying drawing figures, in which like reference numerals identify like elements in the figures, and in which:

FIG. 1 is an illustration of a system for performing pain surveying and visualization in a human body region;

FIG. 2A illustrates a pain rating screen with a 3D human head input, a pain intensity slider, a 3D head rotation control, a set of four anatomical grid controls, and a menu bar;

FIG. 2B illustrates an alternate view of the pain rating screen;

FIGS. 2C-2F illustrate anatomical grid pain rating screens with left, front, back, and right views, respectively;

FIG. 3A illustrates a pain rating screen with a 3D human head with user input pain data;

FIG. 3B illustrates a right side view pain rating screen with user input pain data;

FIG. 4A illustrates a user report age input screen;

FIG. 4B illustrates a user report descriptors input slider screen;

FIG. 4C illustrates a user report duration of attacks input screen;

FIG. 4D illustrates a user report first pain attack input screen;

FIG. 4E illustrates a user report frequency of attacks input screen;

FIG. 4F illustrates a user report impact input slider screen;

FIG. 4G illustrates a user report symptoms and signs input slider screen;

FIG. 4H illustrates a user report triggers input slider screen;

FIG. 5A illustrates a user pain data set load screen;

FIG. 5B illustrates a user pain data set explorer screen;

FIGS. 5C & 5D are alternative views of a user pain data set explorer screen;

FIG. 6A is a an average pain level plot;

FIG. 6B is an average pain area plot;

FIG. 6C is a peripheral nervous system bar graph showing dermatome percentages;

FIG. 6D is a pain characteristics bar graph;

FIG. 6E is a P.A.I.N.S. level plot;

FIG. 6F is an 3D averaging display option control;

FIG. 6G is a 3D human head with a pain change heat map showing change in pain over a loaded data set;

FIG. 6H is a 3D human head with a filled cells average rating heat map;

FIG. 6I is a 3D human head with a simple average rating heat map;

FIGS. 7A-7C are a user summary report;

FIG. 8A is a schema of data representing a patient's user data;

FIG. 8B is a schema of data representing a patient's input pain data;

FIG. 9 is a block diagram illustrating a method for tracking and analyzing pain experienced by a subject;

FIG. 10 is an illustration of a network enabled device for use with the pain tracking and analysis system;

FIG. 11 is a Table of clinical profile data for participants in an example Experiment 1 and Experiment 2;

FIG. 12 illustrates images of a pain aggregation and μ-opioid activation identification process in accordance with Experiment 1;

FIG. 13 is a plot of baseline medial prefrontal cortex receptor density during an ictal migraine phase showing correlation to interictal phase in accordance with Experiment 1;

FIG. 14 illustrates data from an experiment to measure correlations of allodynia levels in accordance with Experiment 2; and

FIG. 15 illustrates images of a pain aggregation and μ-opioid activation for migraine allodynia in accordance with Experiment 2.

### DETAILED DESCRIPTION OF THE PREFERRED EMBODIMENTS

The present application describes techniques for collecting and analyzing a patient's sensed pain information to gather pain intensity, pain location, qualitative pain information. The pain information may be collected from a lifelike rendering of a region of interest, a rendering displayed to the patient and with which the patient may interact to identify locations of pain and the perceived amount of pain. That pain information may be analyzed in a variety of ways to assess a patient's condition and then displayed in various formats for the patient and/or health care professional. The pain information may be collected from a handheld or personal device used by the patient, including cell phones, personal trackers, smart watches, or others, and, in particular, through a mobile device application stored on a common device such as a smartphone.

As a patient's pain symptoms change over time, the present techniques also provide a mechanism for automatically analyzing pain information over time. The techniques may automatically aggregate the pain information and develop a pain score for the patient, a score that may be tracked over time. This pain score is more accurate than conventional techniques and allows for better pinpointing of pain “hotspots” and better tracking of changes in pain “hotspots.” Moreover, however, the present techniques allow for a more accurate assessment of a patient's overall pain levels, or for a single or multiple bodily regions, thereby allowing health care professionals and patient's to better assess pain treatment effectiveness for a particular pain or overlapping pain conditions. Using the more accurate, automated techniques we have been able to evaluate, in vivo, the μ-opioid system during spontaneous episodic migraine headaches and assess variations over patient groups.

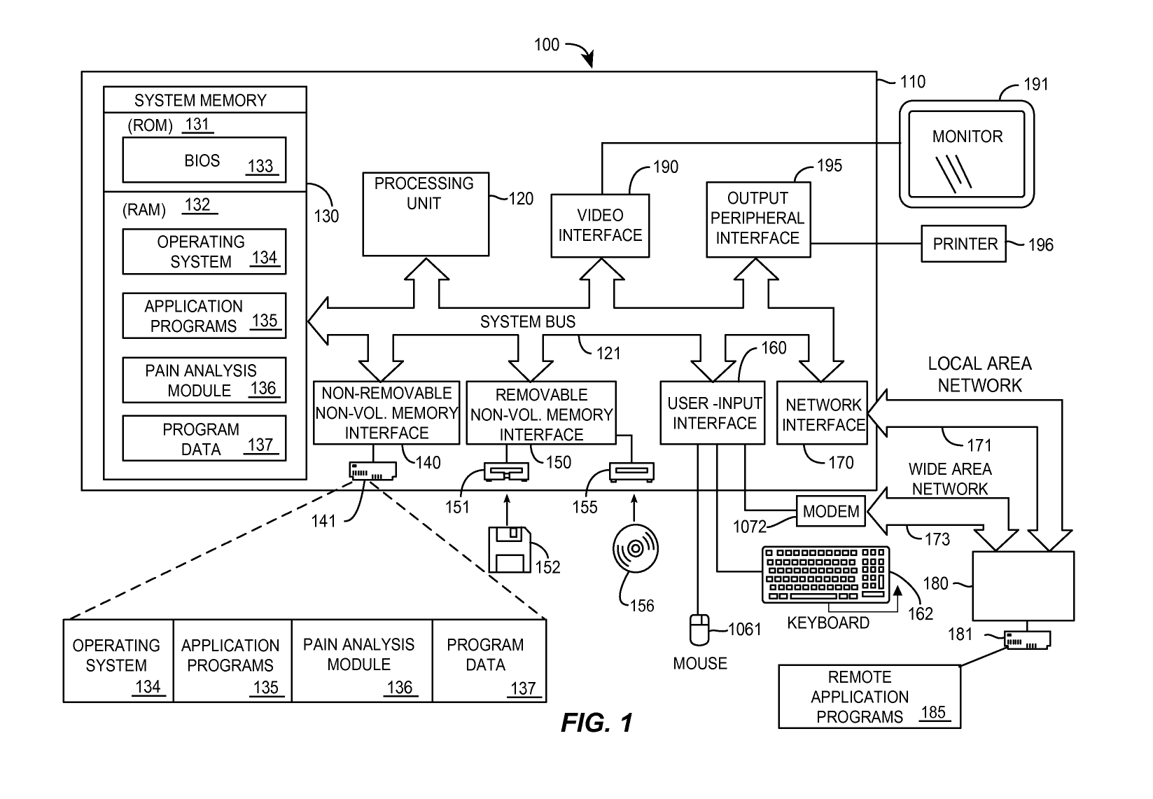

The methods for tracking and analyzing pain experienced by a subject described herein may be implemented in part or in their entirety using one or more computer systems such as the exemplary computer system 100 illustrated in FIG. 1.

Some or all calculations performed in the tracking, analysis, display, transmission, and storage of pain data may be performed by a computer such as the general-purpose computing device in the form of a computer 110, and more specifically may be performed by a processor such as the processing unit 120, for example. In some embodiments, some calculations may be performed by a first computer such as the computer 110 while other calculations may be performed by one or more other computers such as the remote computer 181 in communication with Medical Imaging Device 180. The calculations may be performed according to instructions that are part of a program such as the operating system 134, application programs 135, pain analysis module 136, the program data 137 and/or the remote application programs 185, for example. These programs and modules are shows as residing on hard drive 141 and/or RAM 132. Such functions including, (i) presenting a visual rendering of a bodily region on a device, either connected remotely to the device or formed as part of the computer system 100; (ii) receiving, from a subject interacting with the visual rendering on the display, identified pain data to create one or more pain heat maps; (iii) developing, from the one or more pain heat maps, an aggregated pain data set for the bodily region; and (iv) storing raw data corresponding to one or more pain data sets.

Relevant data may be stored in the ROM memory 131 and/or the RAM memory 132, for example. In some embodiments, such data is sent over a network such as the local area network 171 or the wide area network 173 to another computer, such as the remote computer 181. The networks 171 and 173 may include a variety of hardware for wireless and/or wired communications capabilities. Exemplary wireless communication hardware in the communication networks 171 and 173 may include cellular telephony circuitry, GPS receiver circuitry, Bluetooth circuitry, Radio Frequency Identification (RFID) or Near Field Communication (NFC) circuitry, and/or Wi-Fi circuitry (i.e., circuitry complying with an IEEE 802.11 standard), as well as hardware supporting any number of other wireless communications protocols. The communication networks 171 and 173 may be over wireless or wired communication links. Example wired communications may include, for example, USB circuitry, Ethernet circuitry, and/or hardware supporting any number of other wired communications protocols. The networks 171 and 173 may connect the system 100 to any number of network-enabled devices such as a network-enabled wireless terminal, a phone, a tablet computer or personal digital assistant (PDA), a smartphone, a laptop computer, a desktop computer, a tablet computer, hospital terminal or kiosk, a portable media player, an e-reader, or other similar devices (not shown). Data may be send among the components described herein according to system bus 121 and accepted from a user according to devices connected to user-input interface 160 such as mouse 1061, keyboard 162, modem 1072, or network interface 170.

In some embodiments, the data is sent over a video interface such as the video interface 190 to display information relating to the pain data to an output device such as, the monitor 191, output peripheral device 195, or the printer 196, for example. In other examples, the data is stored on a non-removable non-volatile memory interface 140 such as hard drive 141 or removable non-volatile memory interface 150 such as disc 152 in disc drive 151 or optical disc 156 in optical disk drive 155.

For purposes of implementing the system 100, a patient may interact with the system via a network server, such as a web server communicating via HTTP (hypertext transfer protocol) or any other type of information server capable to transmit information according to any network communications protocol. For example, a patient may access application programs 135 from a remote server, such as using a web-based application, and sending data collected at the patient over a network to the remote server for analysis, visualization, and export.

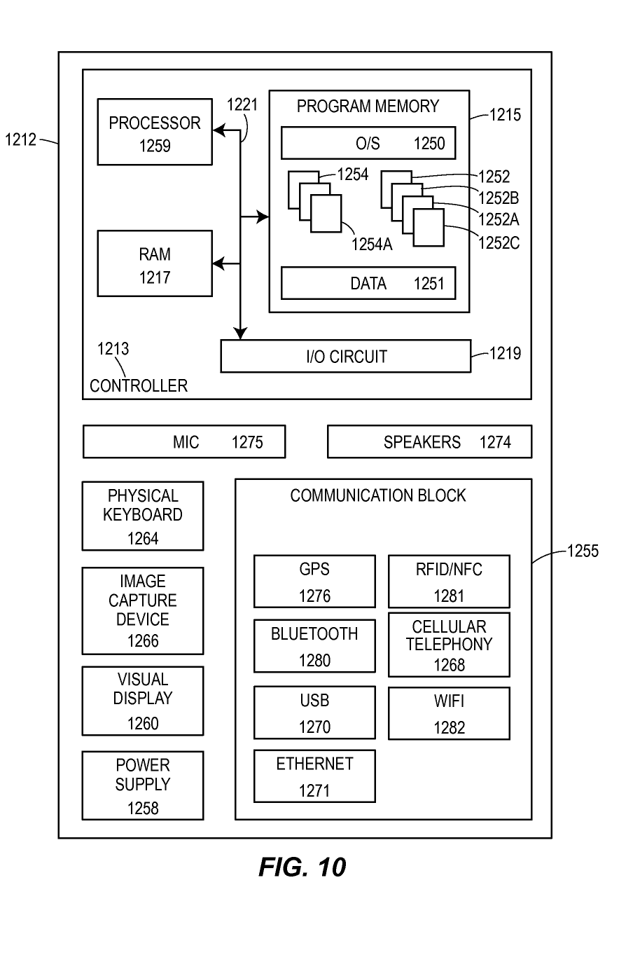

FIG. 10 illustrates an example network-enabled device that maybe used as an implementation of the system 100 to performing pain information collection, display, and analysis. A mobile device 1212 is shown. That mobile device, while described as being a smartphone, may be any type of a network-enabled device, such as a cellular wireless terminal, a phone, a tablet computer or personal digital assistant (PDA), a smartphone, a laptop computer, a desktop computer, a wearable wireless communication device such as a wearable computer, a portable media player, an e-reader, or other similar devices (not shown), as used by a user. Of course, any network-enabled device appropriately configured may interact with the system 100. For convenience, throughout the remainder of this description the system 100 will be described with reference to the device 1212 (i.e., the smartphone). However, it should be understood that, unless otherwise stated, any reference to the device 1212 should be understood as referring to any one of the network-enabled devices.

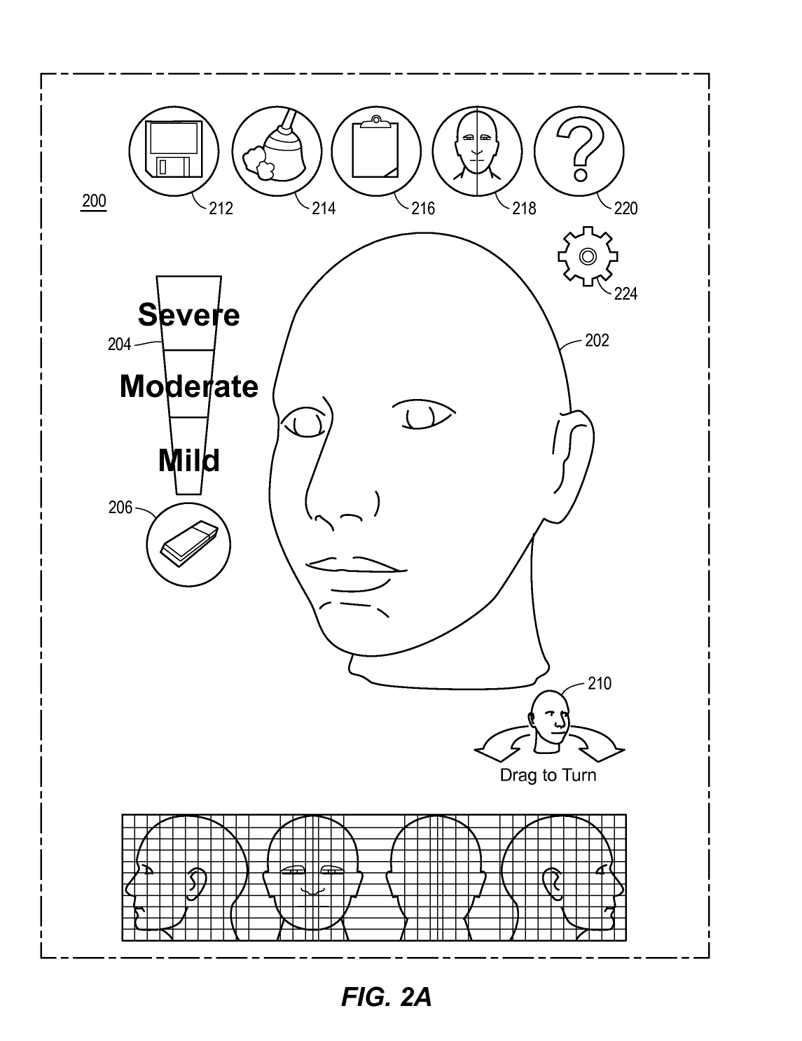

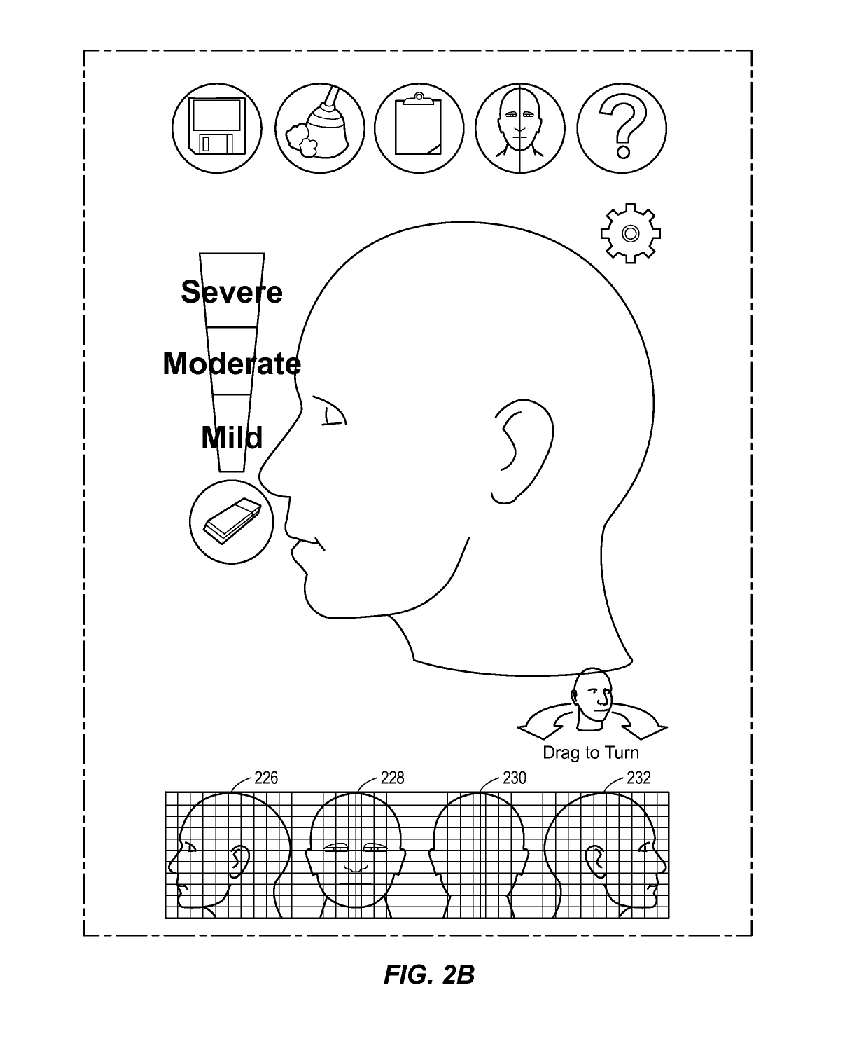

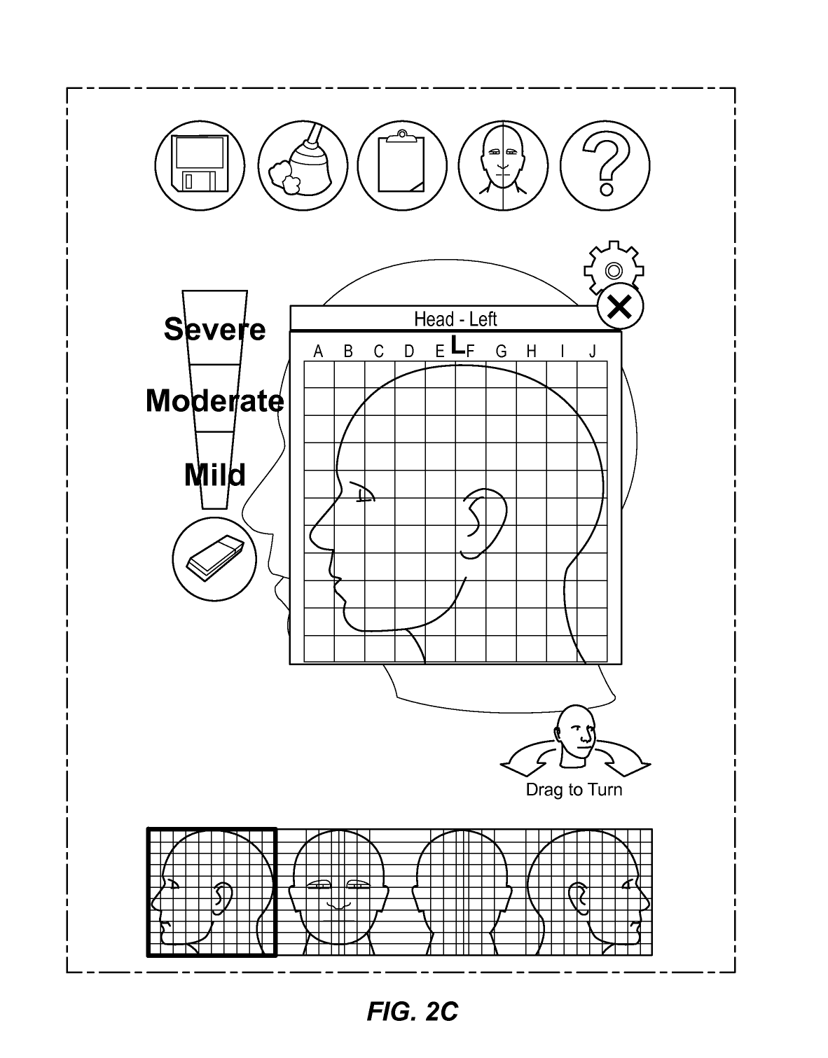

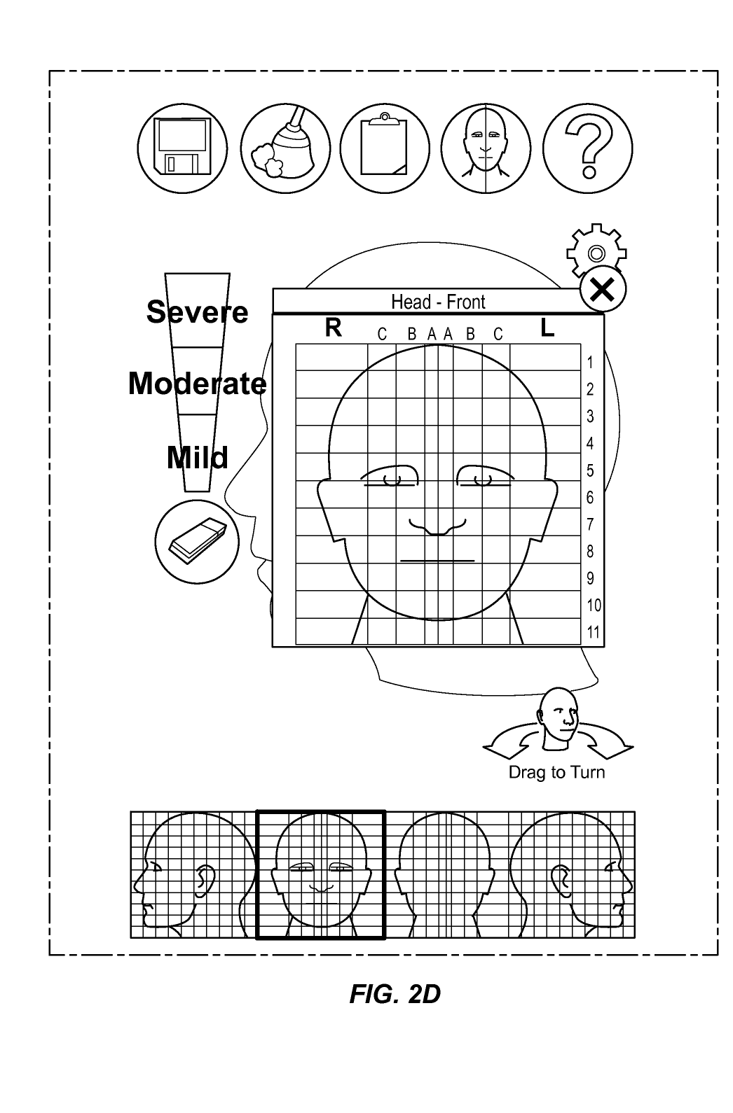

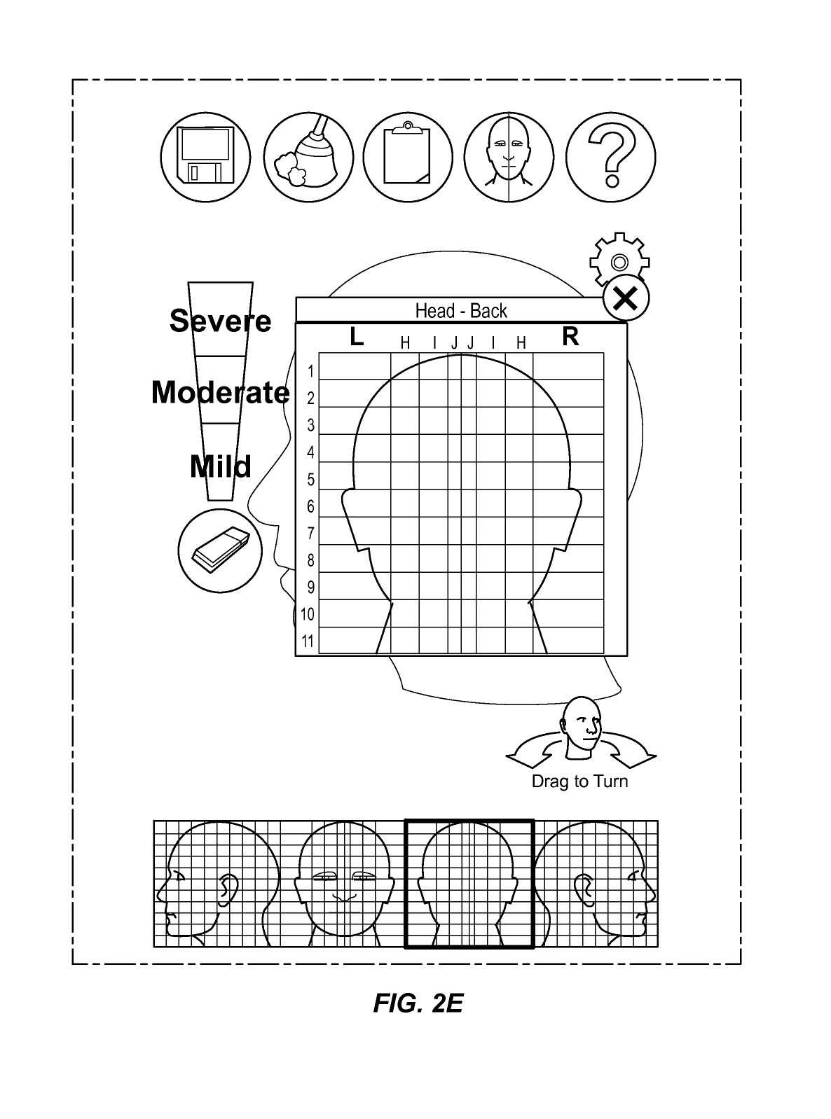

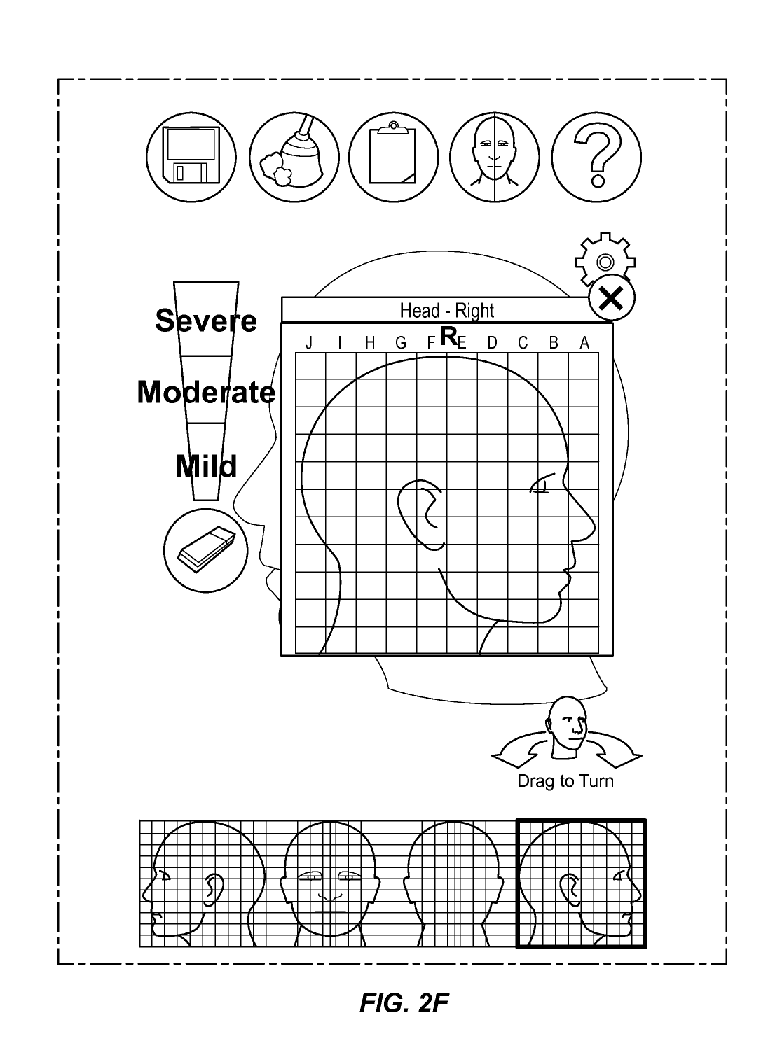

The device 1212 need not necessarily communicate with the network via a wired connection. In some instances, the device 1212 may communicate with the network via wireless signals; and, in some instances, the device 1212 may communicate with the network via an intervening wireless or wired device, which may be a wireless router, a wireless repeater, a base transceiver station of a mobile telephony provider, etc., or other access point. Each of the network-enabled device 1212 may interact with a network access point to receive information including web pages or other information adapted to be displayed on a screen, such as the screens depicted in FIGS. 2-7, for display on the device 1212. Multiple web servers may be provided as well as multiple access points for the purpose of distributing server load, serving different web pages, implementing different portions of the web interface, etc.

The device 1212 may operate in a variety of hardware and/or software configurations. The device 1212 includes a controller 1213. The controller 1213 includes a program memory 1215, a microcontroller or a microprocessor 1259, a random-access memory (RAM) 1217, and an input/output (I/O) circuit 1219, all of which are interconnected via an address/data bus 1221. In some embodiments, the controller 1213 may also include, or otherwise be communicatively connected to, a database (not shown) or other data storage mechanism (e.g., one or more hard disk drives, optical storage drives, solid state storage devices, SIM cards, etc.). It should be appreciated that although FIG. 10 depicts only one microprocessor 1259, the controller 1213 may include multiple microprocessors 1259. Similarly, the memory of the controller 1213 may include multiple RAMs 1217 and multiple program memories 1215. Although FIG. 10 depicts the I/O circuit 1219 as a single block, the I/O circuit 1219 may include a number of different types of I/O circuits. The controller 1213 may implement the RAM(s) 1217 and the program memories 1215 as semiconductor memories, magnetically readable memories, and/or optically readable memories, for example.

The program memory 1215 and/or the RAM 1217 may store various applications (i.e., machine readable instructions in a non-transitory form) for execution by the microprocessor 1259. For example, an operating system 1250 may generally control the operation of the device 1212 and provide a user interface to the device 1212. Various applications 1254 may allow the user to perform various functions associated with the device 1212. By way of example, and without limitation, the applications 1254 may include, among other things: an application for accessing telephony services; an application for sending and/or receiving email; an application for sending and/or receiving text or short message service (SMS) messages; a calendar application; a contact list application; a web browsing application; etc. In particular, the applications 1254 may include an application 1254A for capturing electronic document data associated with system 100.

The program memory 1215 and/or the RAM 1217 may also store a variety of subroutines 1252 for accessing specific functions of the device 1212. By way of example, and without limitation, the subroutines 1252 may include, among other things: a subroutine 1252A for accessing geolocation services, a subroutine 1252B for accessing image capture services, and other subroutines 1252C, for example, implementing software keyboard functionality, interfacing with other hardware in the device 1212, etc.

The program memory 1215 and/or the RAM 1217 may further store data 1251 related to the configuration and/or operation of the device 212, and/or related to the operation of one or more of the applications 1254 or subroutines 1252. For example, the data 1251 may be image data captured by an image capture device, may be data input by a user, may be data received from a server, data determined and/or calculated by the processor 1259, etc. In addition to the controller 1213, the device 1212 may include other hardware resources. For example, the device 1212 may include a power supply 1258, which may be a battery in the case of a mobile device. The device 1212 may also include various types of input/output hardware such as a visual display 1260, a physical keyboard 1264, an image capture device 1266, one or more speakers 1274, a microphone 1275, and/or a pointing device (not shown). In an embodiment, the display 1260 is touch-sensitive, and may cooperate with a software keyboard routine as one of the software routines 1252 to accept user input.

The device 1212 may be configured with a communication block 1255 including a variety of hardware for wireless and/or wired communications. Example wireless communication hardware in the communication block 1255 may include cellular telephony circuitry 1268, GPS receiver circuitry 1276, Bluetooth circuitry 1280, Radio Frequency Identification (RFID) or Near Field Communication (NFC) circuitry 1281, or Wi-Fi circuitry 1282 (i.e., circuitry complying with an IEEE 802.11 standard), as well as hardware supporting any number of other wireless communications protocols. Example wired communications hardware in the communication block 1255 may include, for example, USB circuitry 1270, Ethernet circuitry 1271, and/or hardware supporting any number of other wired communications protocols.

It should be recognized that different mobile devices may implement different mechanisms for user input. In an example described above, the device 1212 may have a touch sensitive display screen 1260. Accordingly, “buttons” which are displayed on the screen and are not physical buttons, are “pressed” by touching the screen in the area of the button. However, those of ordinary skill in the art will readily appreciate that such user interface controls may be accomplished in other manners, such as using soft-keys, navigating controls using navigation buttons on a keyboard or using a roller ball, selecting numbers corresponding to different controls, entering information on a keyboard, etc. Additionally, the device 1212 may receive voice commands via the microphone 1275. Such voice commands may be interpreted by an application 1254 (e.g., the Siri® product from Apple Computer).

It should be understood that it may be desirable for some or all of the data transmitted from the system server to the device 1212, or vice versa, to be encrypted and/or otherwise transmitted in a secure manner (e.g., using Hypertext Transfer Protocol Secure, known as “HTTPS” or another secure communications protocol).

Typically, a user may launch or instantiate a user interface application (e.g., a web browser, mobile application, or other client application) from a network-enabled device, such as device 1212 to establish a connection with the system 100. In this way, the system 100 may be implemented on a server.

The computer system 100 and/or mobile device 1212 may be used to create a system for collecting, displaying, and analyzing pain information.

In an example implementation, the bodily region of interest is the head of a patient. To pinpoint locations of pain with the head, we have developed a series of mapping protocols. The head may be divided into cells (i.e., sub-regions) using a square grid system with vertical and horizontal coordinates with reference to anatomical landmarks. In one example, the head is mapped with columns A-J starting at the front of the head and moving to the back when viewed in profile, and rows 1-11 starting at the top of the head and moving down to the neck when the head is viewed from any direction. A set of columns A-J is applied separately to each hemisphere of the head, left and right, such that there is a set corresponding to each side. The term “cell” within this description is used to denote one element of the square grid that may be represented in a three-variable coordinate system including a column, a row, and a hemisphere, e.g., B/4/L denotes the second column, fourth row, on the left hemisphere of the head. The location of the columns and rows are chosen with reference to anatomical landmarks. For instance, the line between rows 5 and 6 is set at the center point of the eyes; the line between rows 7 and 8 is set to be the inferior side of the nose; the line between rows 10 and 11 is the inferior side of the chin; the line between columns B and C is the center point of the eyes. More examples will be clear with reference to the anatomical grids shown in FIGS. 2C-2F below. Is this way, any cell may be located with a 3-tuple coordinate, and may be referenced by a patient experiencing pain regardless of differences between the anatomical features of the patient's head and the head model as shown herein.

Once the device has stored one or more pain data sets for a patient, these data are available for a variety of display options. Pain analysis module 136 may display data on the 3D head projection image, either as single pain data sets in a selectable list, or showing aggregate data across selected pain data sets such as averages, frequencies or change in pain as described in more detail below. The data may be presented according to a dermatome calculation based on the indicated pain locations. The selected pain data sets may be exported in data formats, as shown in more detail below, to any of a variety of statistical analysis programs such as Microsoft Excel, SPSS, Stata, SigmaStat, Mathematica, and more. In this way, any set or sets of pain data from a single patient or any number of patients may be analyzed and visualized according to the invention.

Referring now to FIG. 2A, a pain rating screen 200 is illustrated. Pain rating screen may be rendered on either computer system 100 by pain analysis module 136 or mobile device 1212, as may all screens referred to in FIGS. 2-7. Pain rating screen 200 displays a 3-dimensional head projection 202, pain intensity slider 204, and cell eraser 206. Head rotation control 210 facilitates manipulation of the 3D model head. Pain rating screen 200 further contains menu buttons for input and management of pain and user information including save button 212, clear button 214, user report button 216, pain information analysis button 218, help button 220, and settings button 224. FIG. 2B shows the pain rating screen of FIG. 2A wherein the head has been rotated to the right into profile view via user interaction with head rotation control 210. Pain rating screen 200 displays four anatomical grid thumbnail displays 226, 228, 230, and 232, selectable by the user. FIGS. 2C-2F illustrate the enlarged anatomical grids that are displayed when the user selects the corresponding thumbnail. The use of anatomical landmarks in addition to those described above will be apparent with reference to FIGS. 2C-2F such as the line between columns E and F, shown here as a centerline, on the superior side of the ear on FIG. 2C or the line between rows 5 and 6 on the superior side of the ears as shown in FIG. 2D.

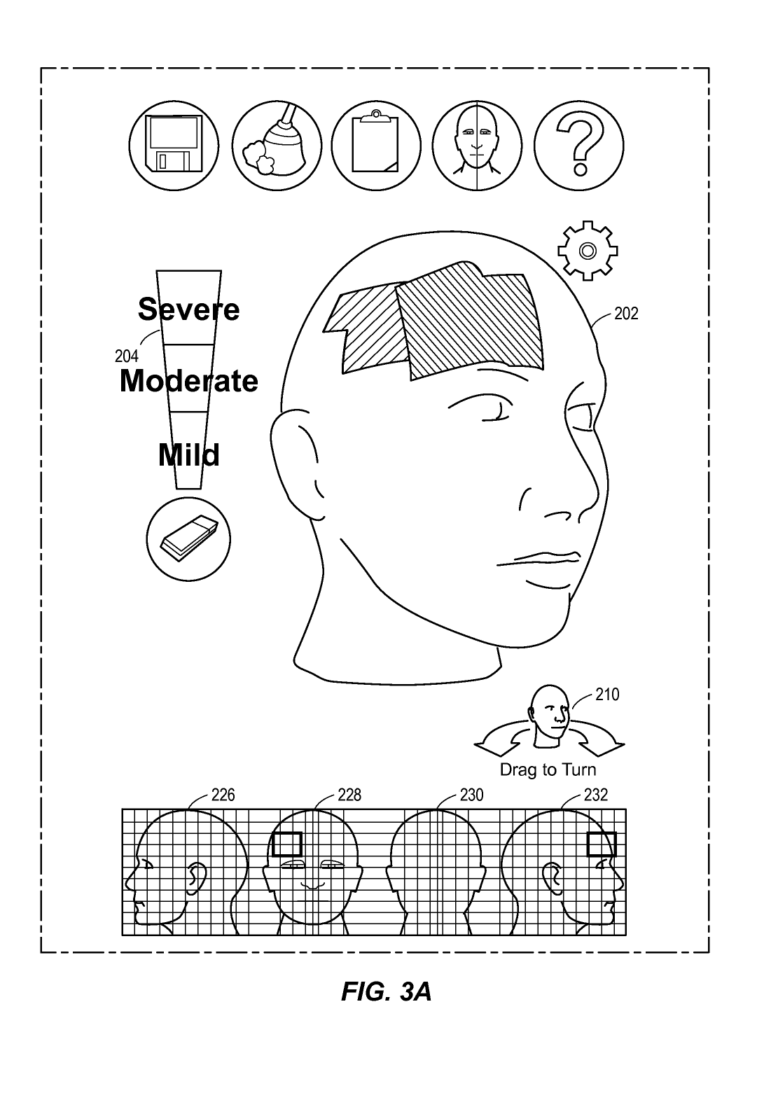

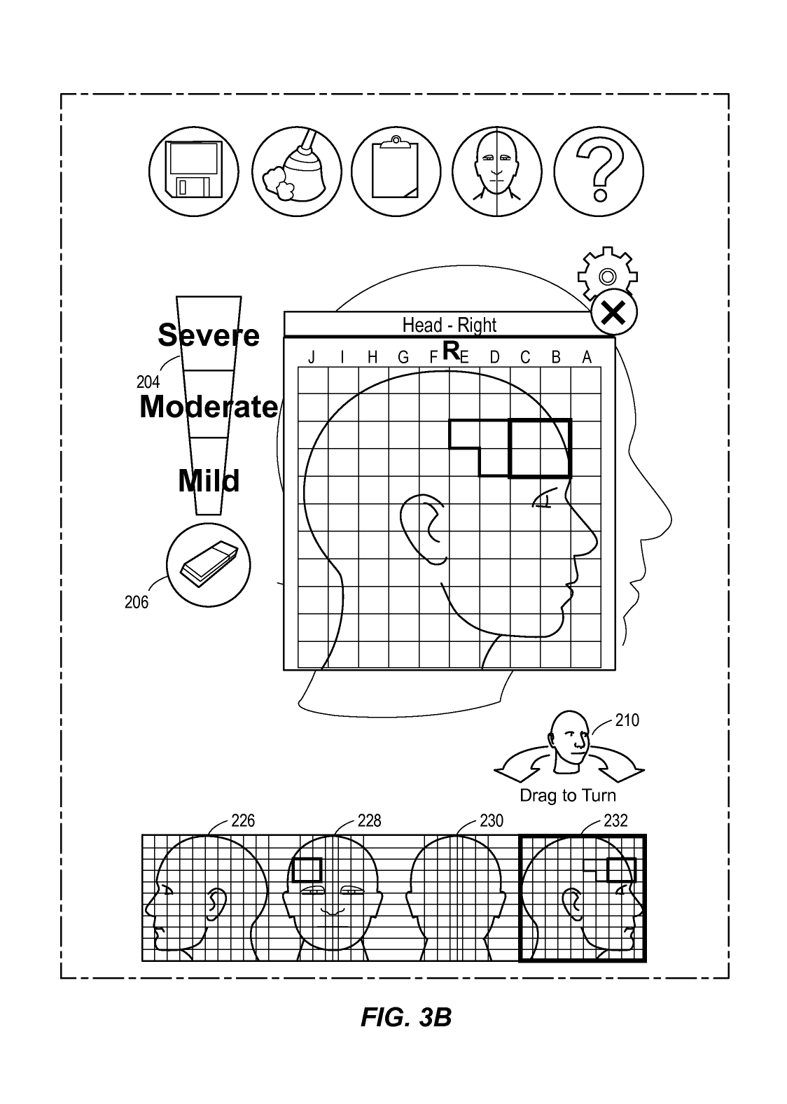

Referring now to FIG. 3A, patient entry of pain information is shown on a pain rating screen rendered by pain analysis module 136 or on mobile device 1212. A patient may select a pain intensity level from 1-3 corresponding to mild, moderate, or severe from pain intensity slider 204, and select or “paint” any desired cells on 3D head projection 202. In FIG. 3A, the user has selected cells from columns B and C and rows 6 through 9 on the right hemisphere as mild pain intensity, cells from columns D and E on the right hemisphere at rows 3 to 4 as mild pain intensity, as well as a 2×2 block of cells in columns B and C, rows 3 to 4 as severe intensity. The patient may interact with the resulting pain heat map on 3D head projection by rotating the via head rotation control 210 or by erasing previously selected cells via cell eraser 206. The patient may select any of anatomical grid thumbnails 226, 228, 230, and 232 to expand the associated anatomical grid for further pain cell selection. An example is shown in FIG. 3B of user selection of anatomical grid thumbnail 232, corresponding to right side view of the head. The user may make further selections on this view using pain intensity slider 204 or cell eraser 206, and by tapping the desired cells to “paint” them with pain data in the view presented by FIG. 3B.

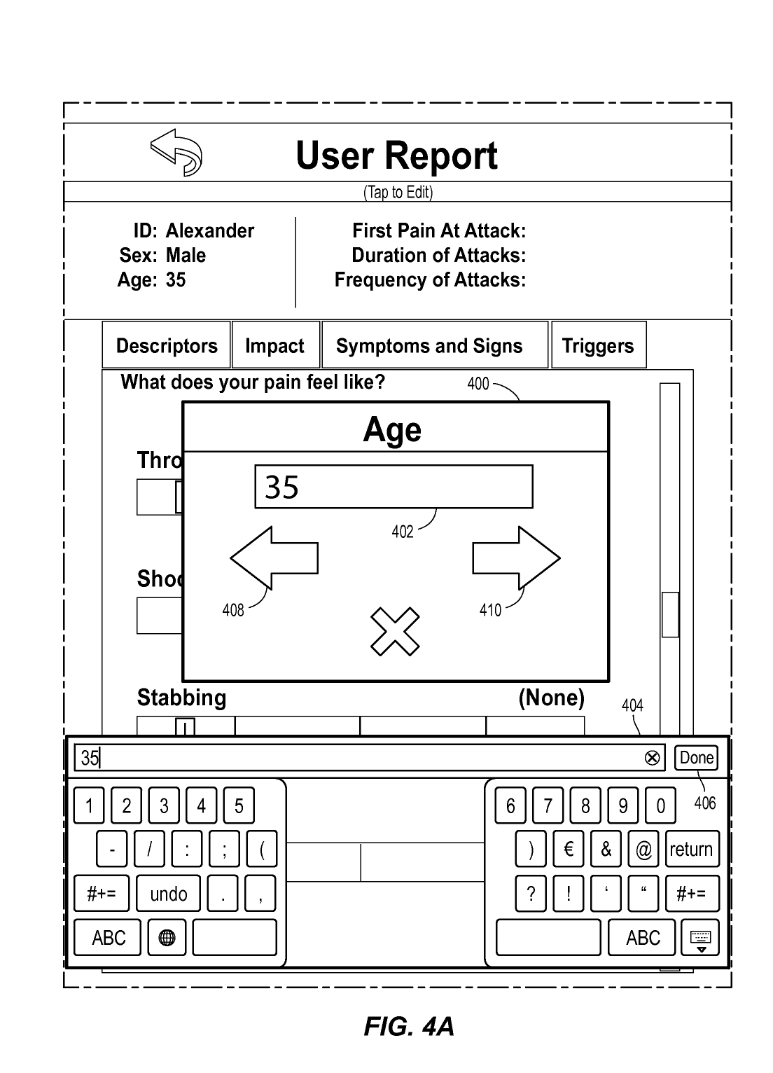

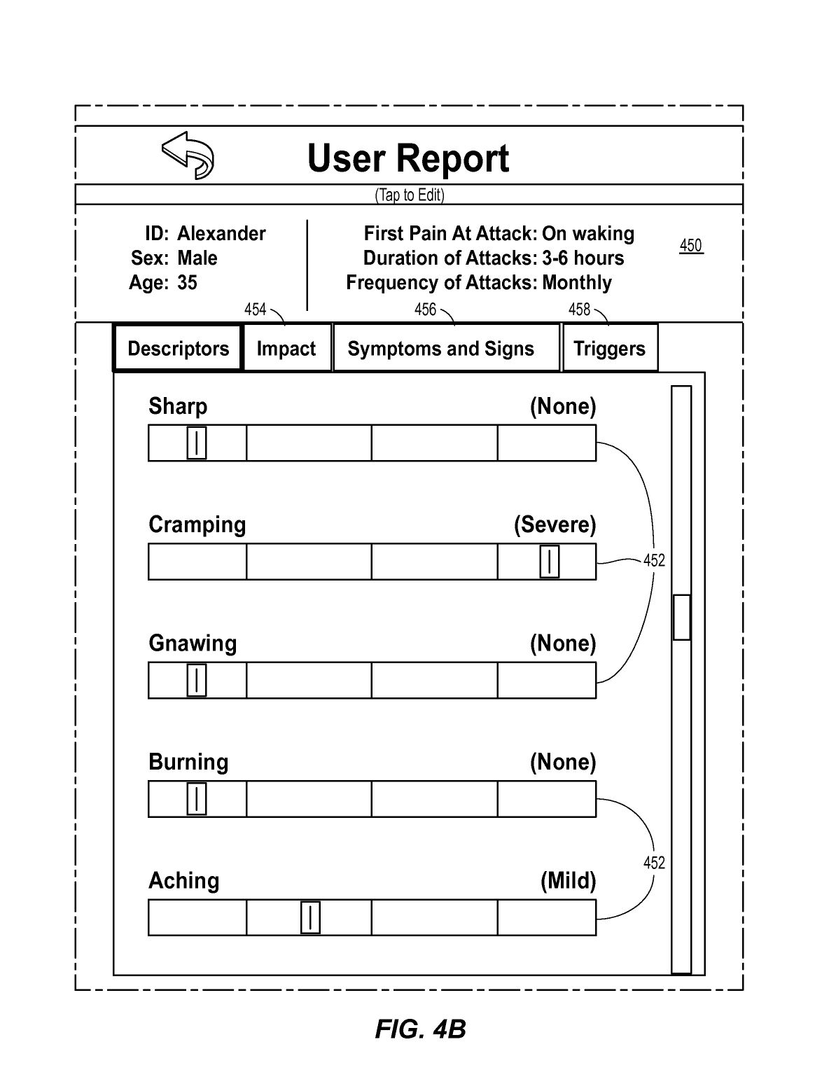

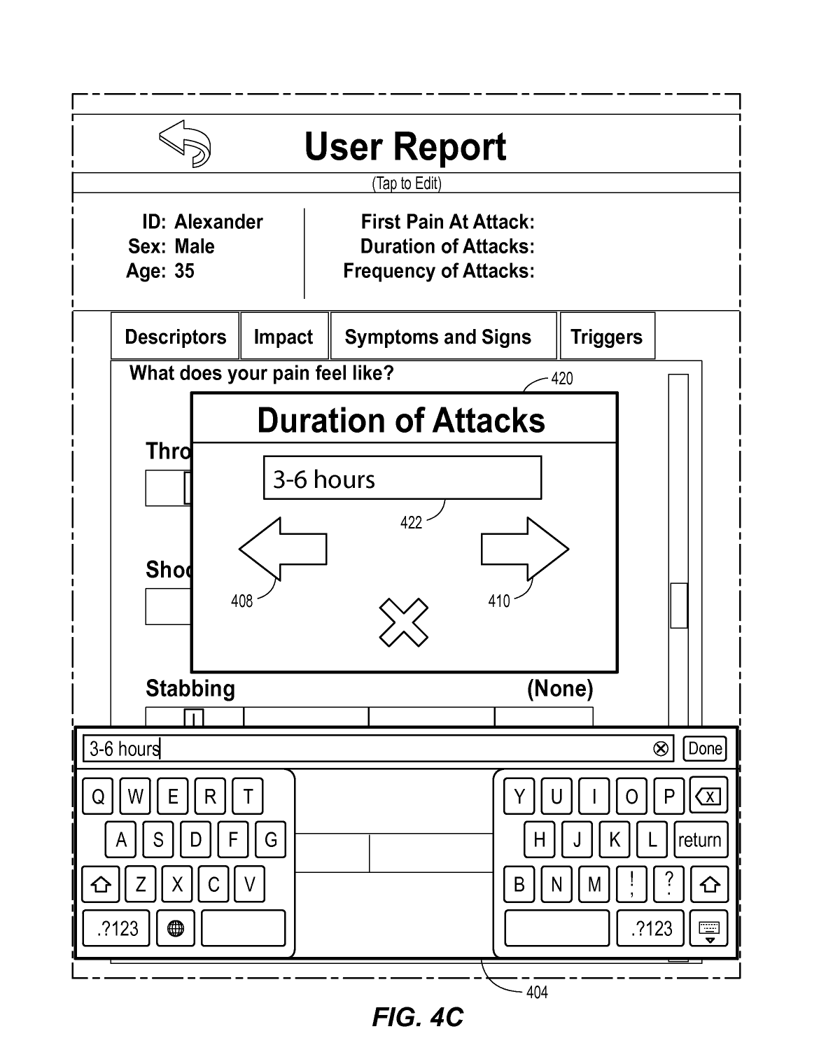

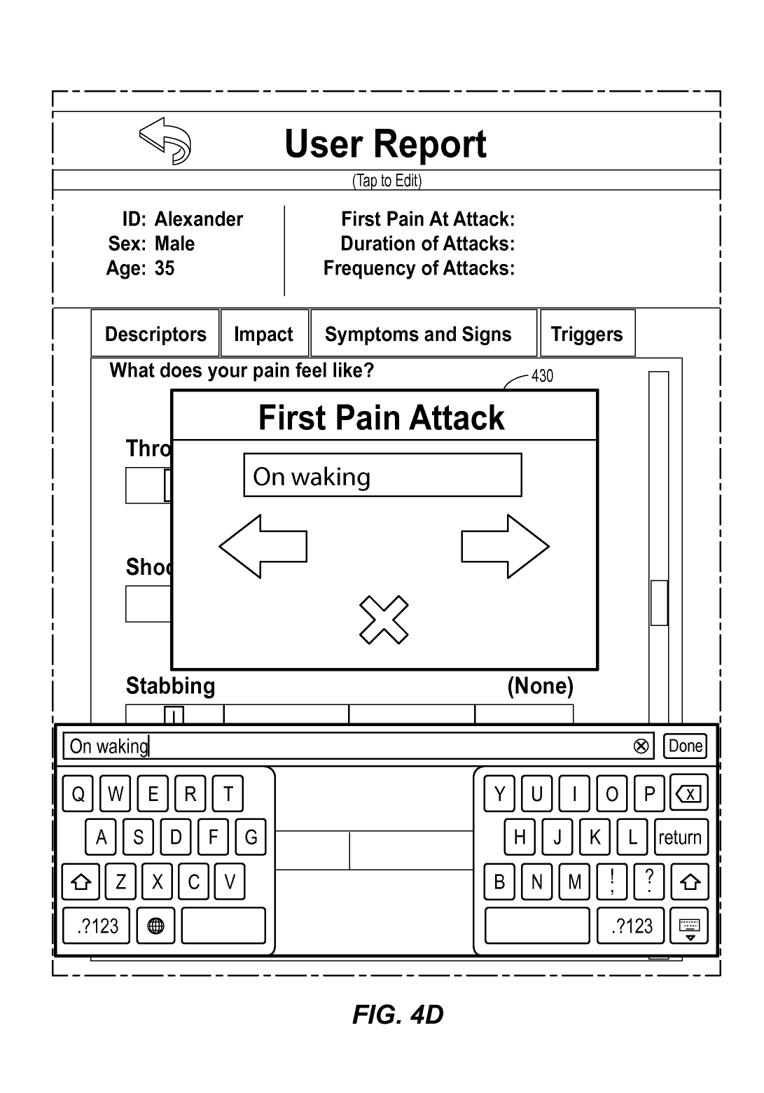

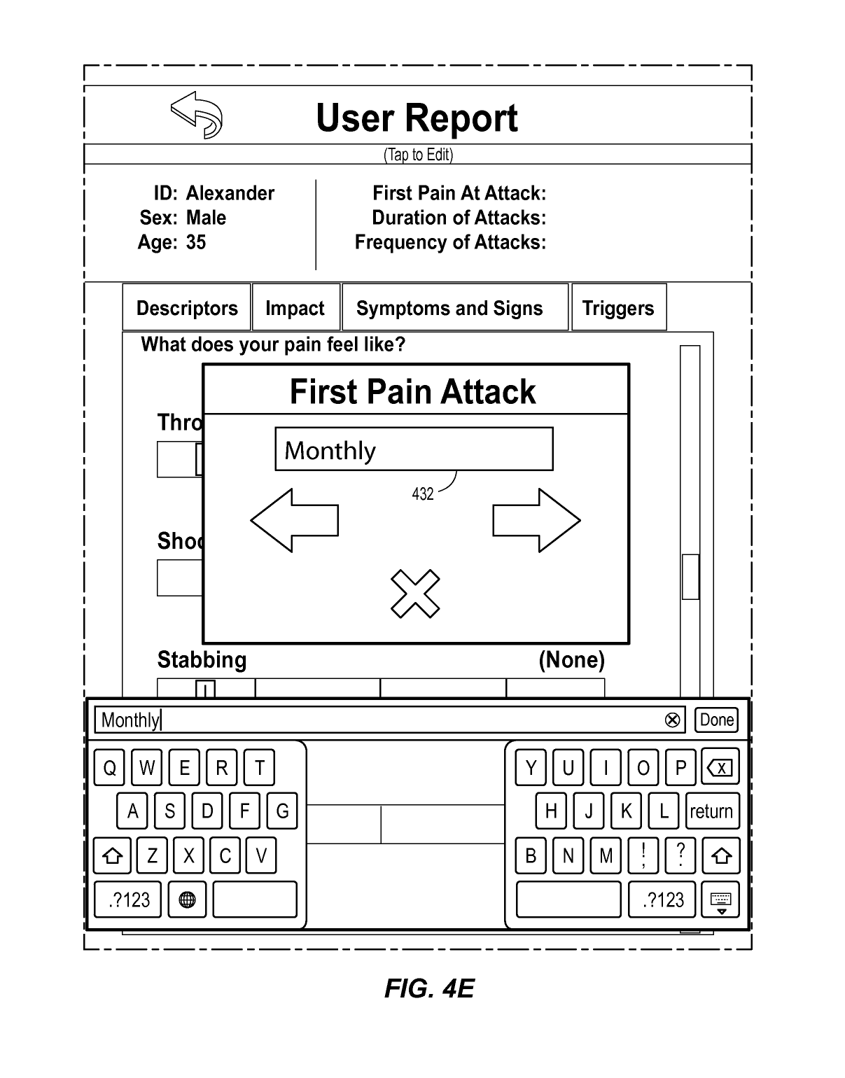

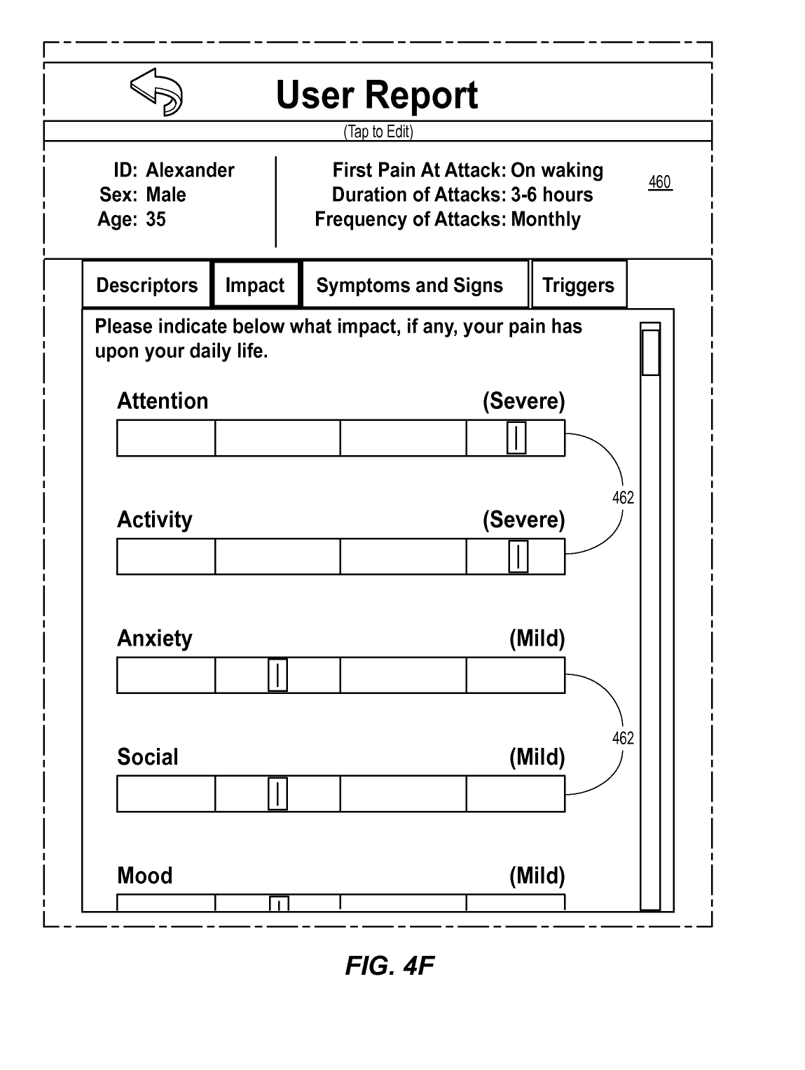

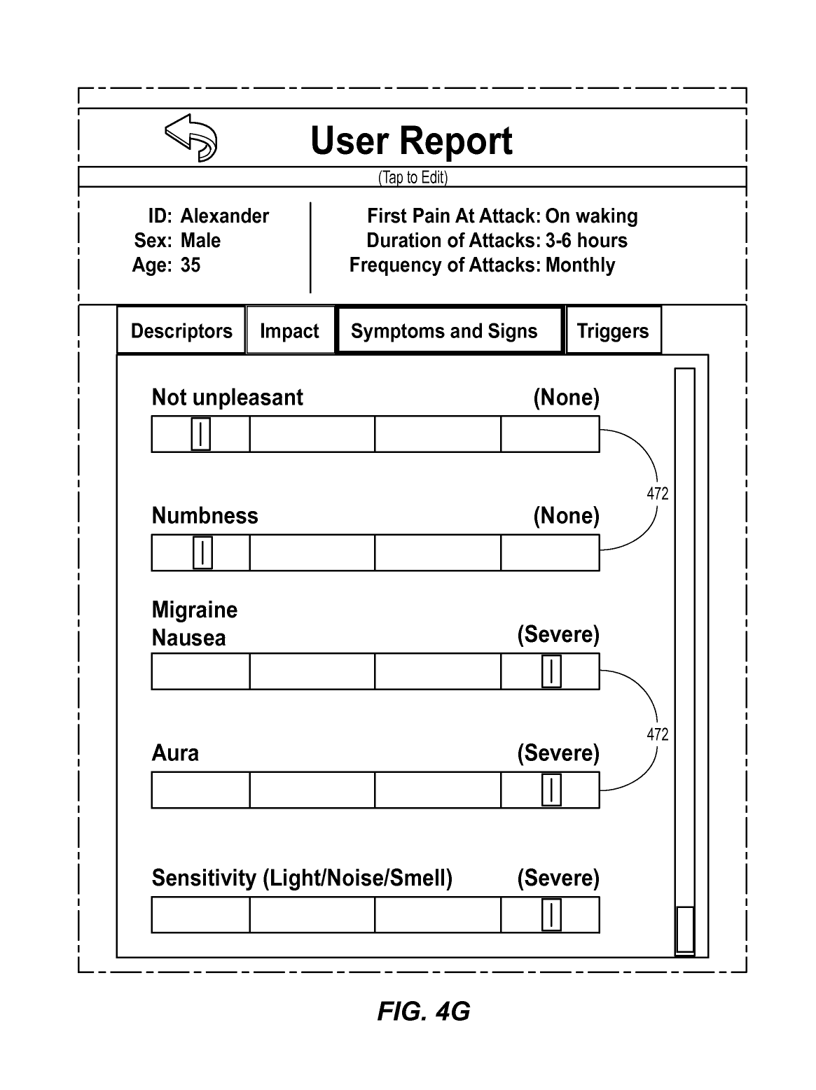

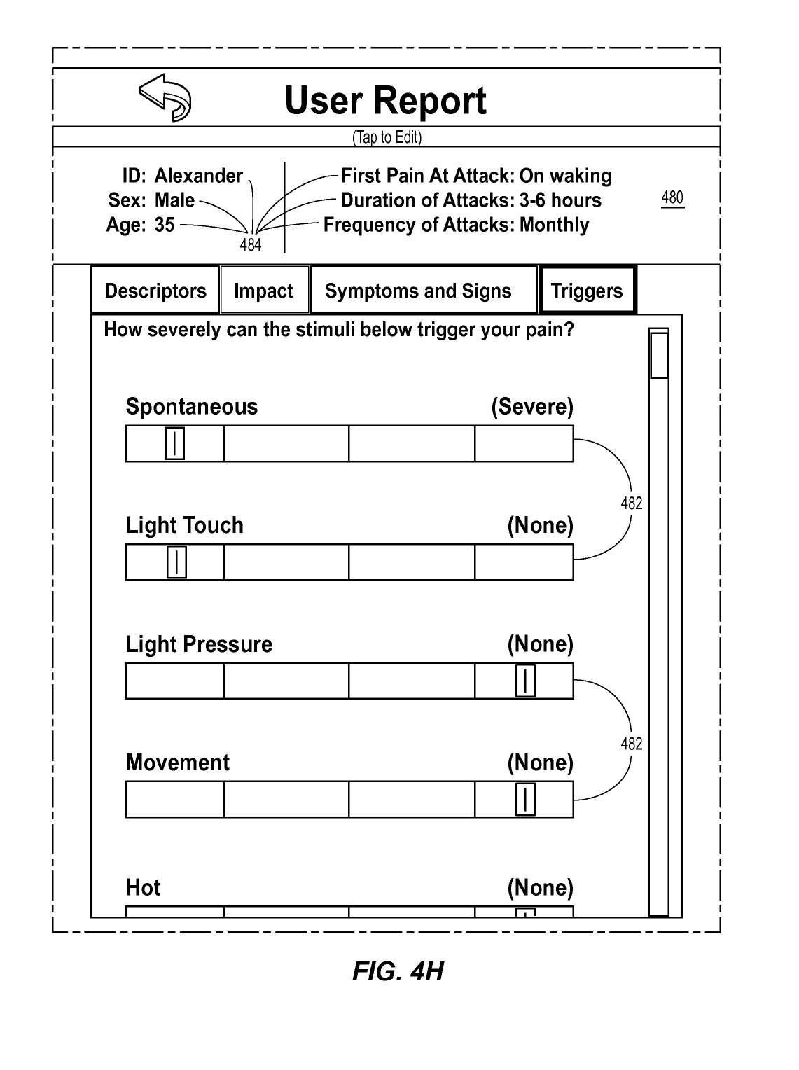

Referring again to FIG. 2A, there is shown a group of menu buttons 212, 214, 216, 218. Selection of user report menu button 216 causes display of User Report screens shown in FIGS. 4A-4H for collection of user demographic data and qualitative pain data. In FIG. 4A, the user is presented with an age input screen 400 containing age input box 402 selectable via software keyboard 404. Age input screen 400 may be rendered on either computer system 100 or mobile device 1212, as may all screens referred to in FIGS. 2-7. The user may complete entry of age information using Done button 406 or via navigation arrows 408 and 410. User navigation via navigation arrow 410 presents the user with duration of attack entry screen 420 as illustrated in FIG. 4C. The user here may enter a text string descriptive of his perceived attack duration into text box 422 using software keyboard 404. The user may again navigate away from this screen using navigation arrows 408 and 410. Similarly, first pain attack entry screen 430 is presented when the user navigates via navigation arrow 410 as illustrated in FIG. 4D. Frequency of attack screen 440, as illustrated in FIG. 4E, may be navigated to using navigation arrow 410, and allows user entry of a descriptive string relating to the frequency of his attacks in text box 432 using software keyboard 404.

FIG. 4B illustrates pain descriptor screen 450, which presents the user with a series of descriptor sliders 452 that measure the respective qualities on a 0-3 scale corresponding to none, mild, moderate, and severe, respectively. Pain descriptors may include, but are not limited to: throbbing, shooting, stabbing, sharp, cramping, burning, aching, heavy, tender, splitting, exploding, massive, and pounding, or any other terms known in the art to describe sensed pain. Also shown in FIG. 4B are user report tabs 454, 456, 458 corresponding to the associated qualitative pain information entry screens as described herein. User report impact tab 454, is selectable by the user and presents the impact slider screen 460 as shown in FIG. 4F. Impact slider screen 460 is analogous to pain descriptor screen 450 in that it presents the user with a series of slider inputs 462 corresponding to respective qualities of the user's sensed pain that are rated on a 0-3 scale corresponding to none, mild, moderate, and severe, respectively. Slider inputs 462 may correspond to qualities including, but not limited to: attention, activity, anxiety, social, mood, sleep, or any other qualities known in the art to be impacted by a user's sensed pain level. Similarly, Symptoms and Signs tab, when selected by a user, presents symptoms sliders 472 corresponding to symptoms grouped as follows and rated according to the 0-3 scale as illustrated in FIG. 4G: inflammatory: swelling, redness, heat; abnormal sensation: unpleasant, not unpleasant, numbness; Migraine: nausea, aura, sensitivity to light, noise, or smell. Finally, Triggers tab, when selected by a user, presents triggers sliders 482 as shown in FIG. 4H and includes, but is not limited to: spontaneous, light touch, light pressure, movement, hot, and cold. User report slider screens as described herein further permit the user to select any of the text screen entry screens via selection of text entry field displays 484 in the same manner as when navigated to using navigation arrows 408 and 410 as described above.

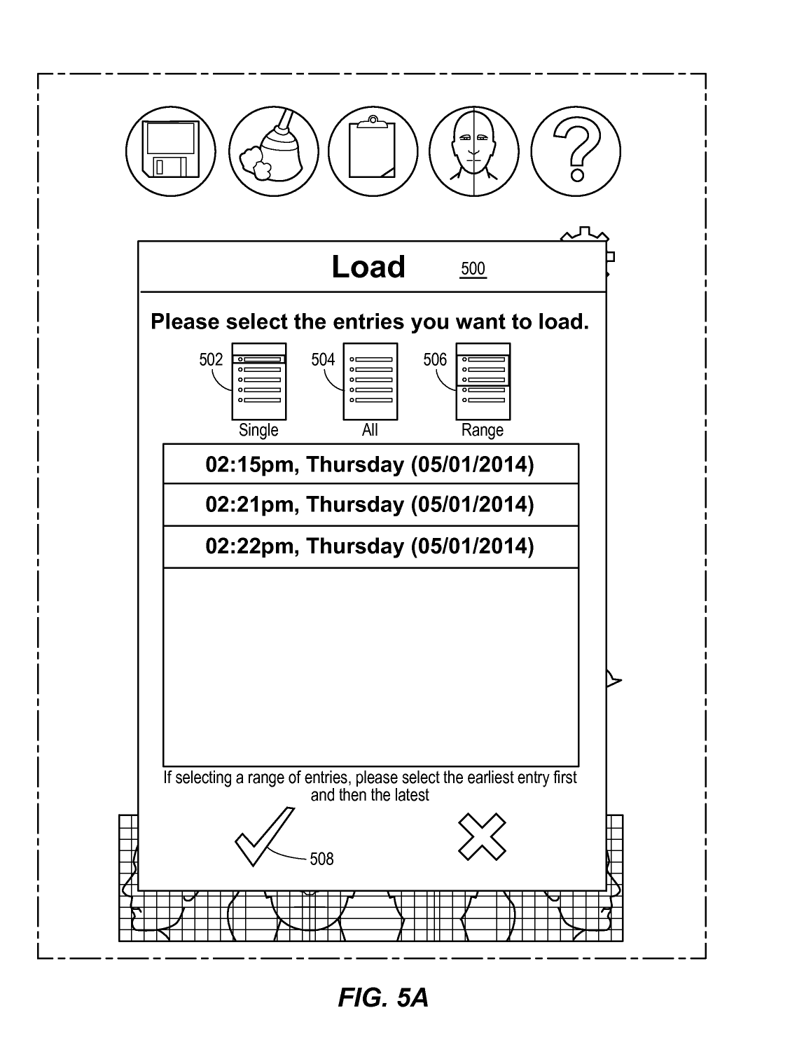

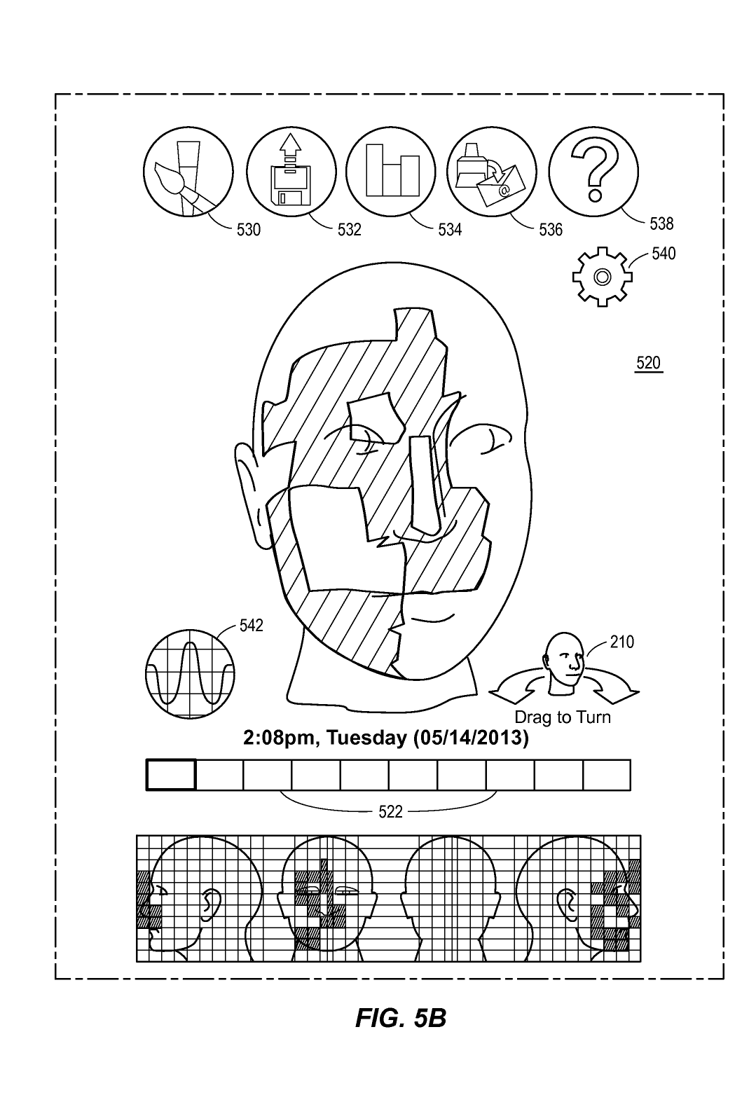

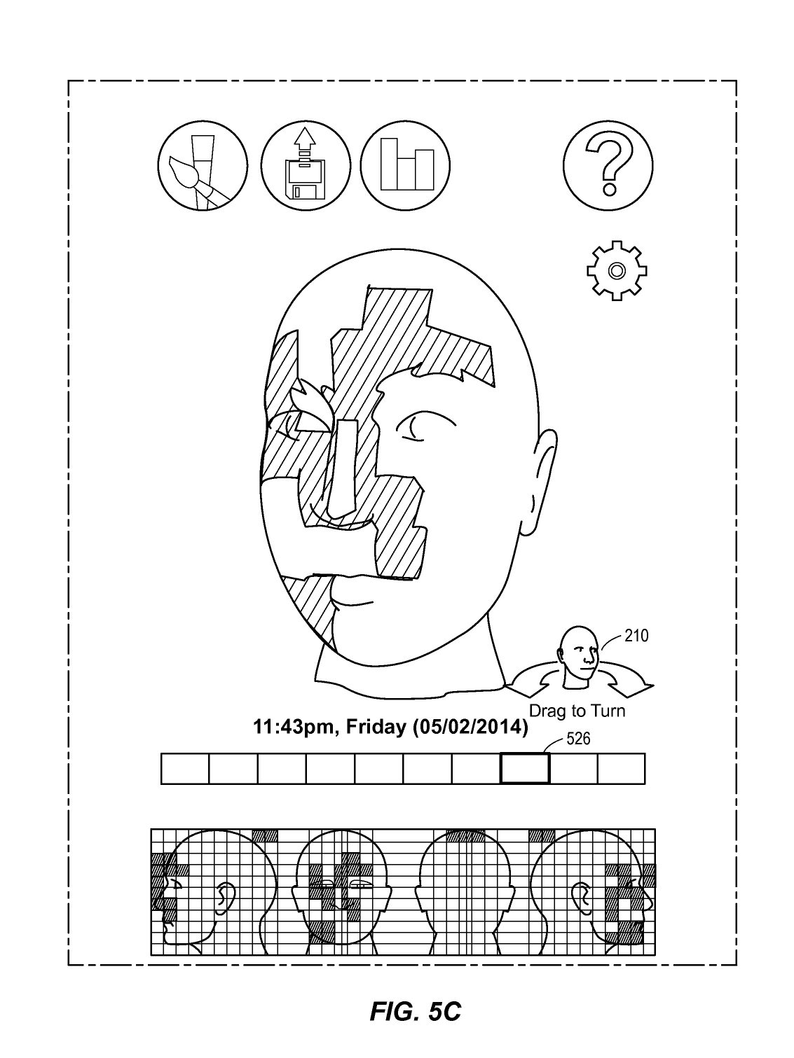

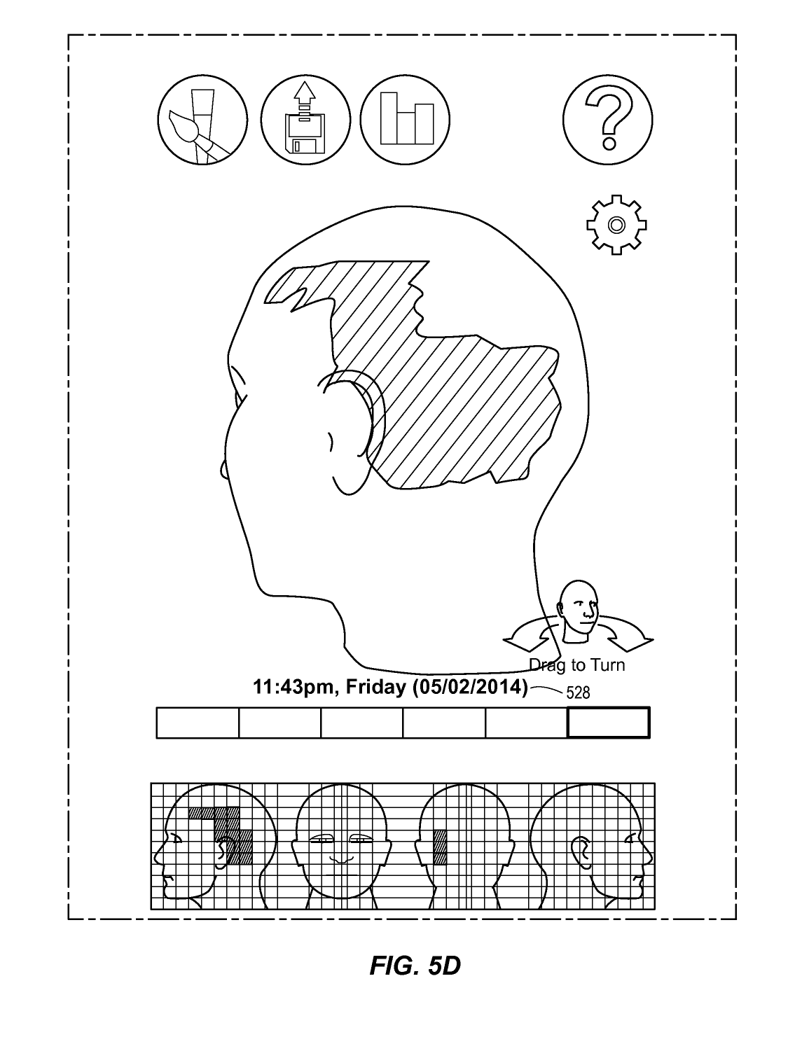

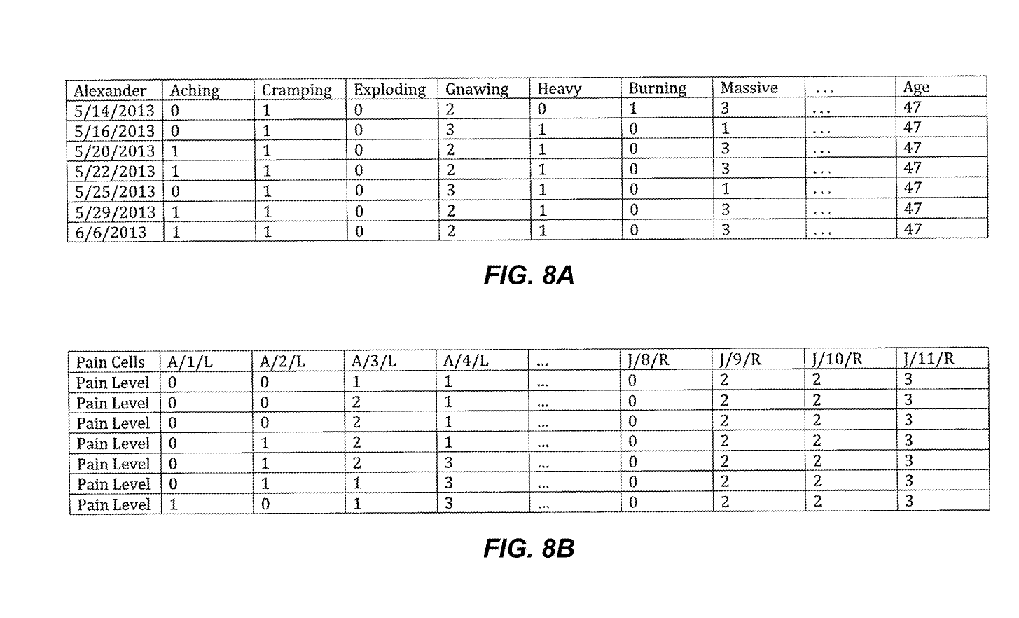

Referring again to FIG. 2A, there is presented to the user save button 212. Selection of this button by the user permits storage of all entered pain location, pain intensity, and descriptive pain information as described in FIGS. 2-4 as a pain data set. Selection of user save button 212 further associates each patient's stored pain data set with a date and timestamp. The manner of storage and data format of this data is described in further detail herein with reference to FIGS. 8A and 8B. Once one or more user pain data sets have been stored, they may be selected according to load screen 500 shown in FIG. 5A. The user may select among selection options 502, 504, 506 to load a single pain data entry, all stored pain data entries, or a range of pain data entries, respectively. The user may then activate checkmark 508 to load the selected pain data entries. FIG. 5B is a screenshot of loaded pain data screen 520. This screen permits visualizations of single pain data sets or of aggregate analysis and visualizations of multiple pain data sets. Data set selection control 522 comprises n bars where n is the number of pain data sets loaded in load screen 500 shown in FIG. 5A. The selected bar of data set selection control is indicated by highlighting focus and by display of timestamp 524 on loaded pain data screen 520. For each selected pain data set, 3D head figure displays that set's data, which may be manipulated according to 3D head figure rotation control 210. Similarly, for each selected pain data set, anatomical grid thumbnails 226, 228, 230, 232 indicate the selected pain locations and intensity levels according to the selected data set. FIGS. 5C and 5D illustrate load screen 520 with different pain data sets loaded according to data set selection control with timestamps 526 and 528.

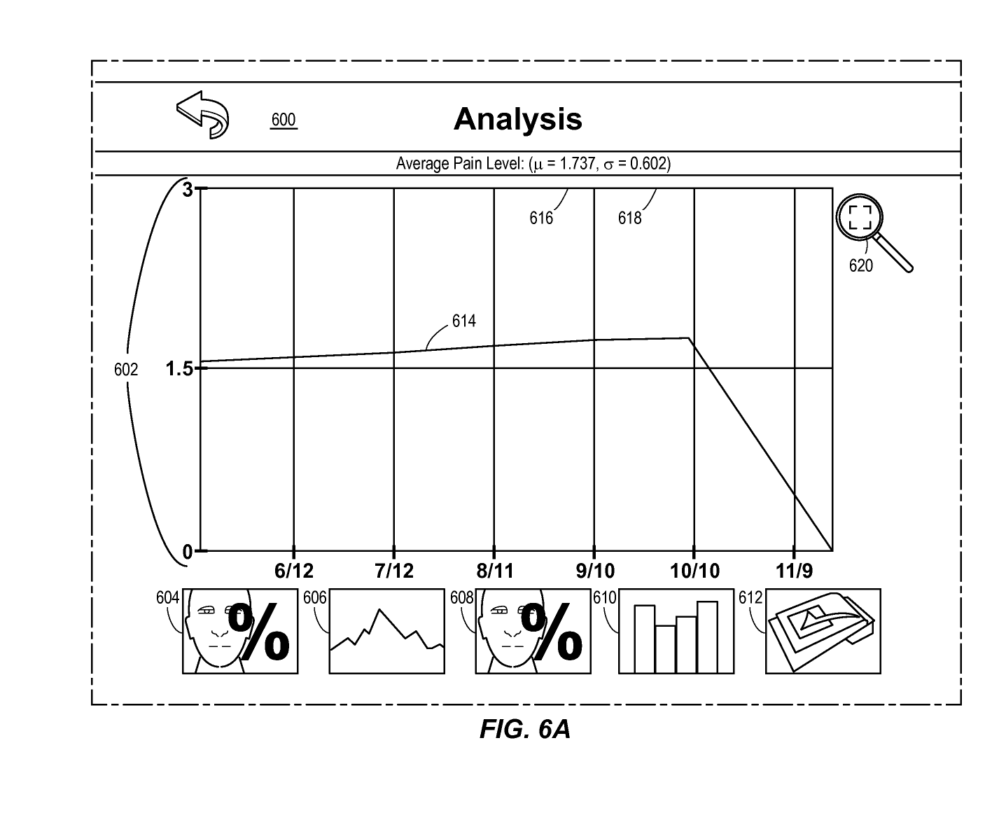

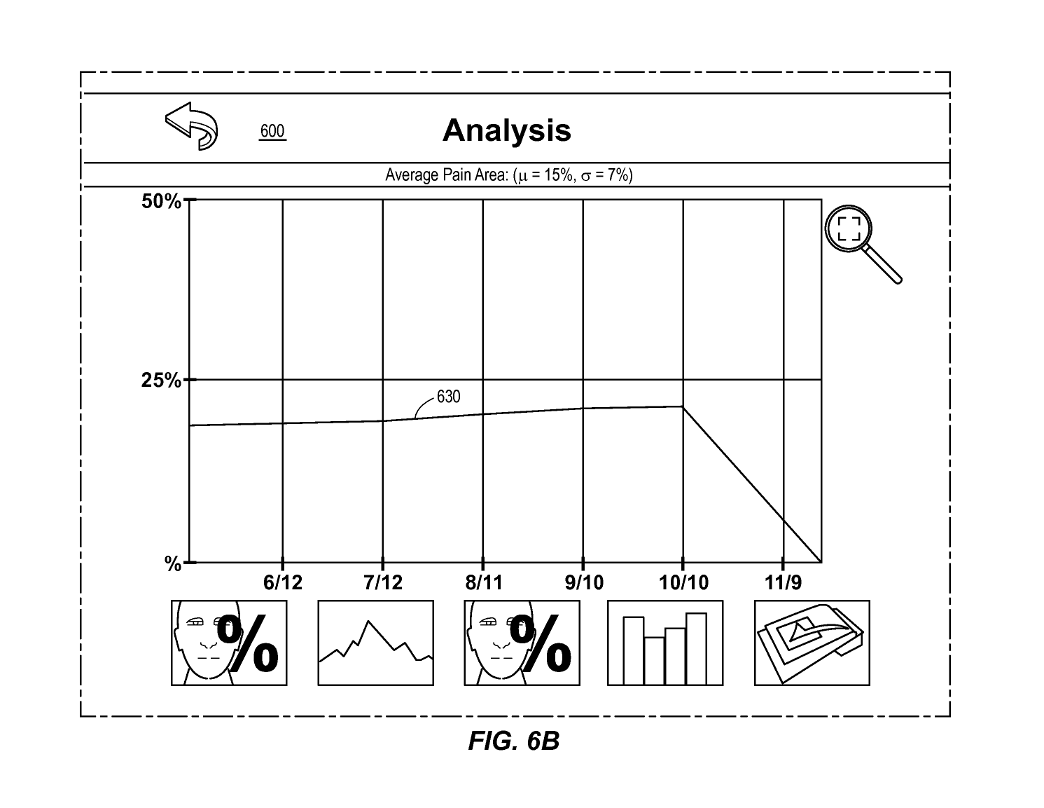

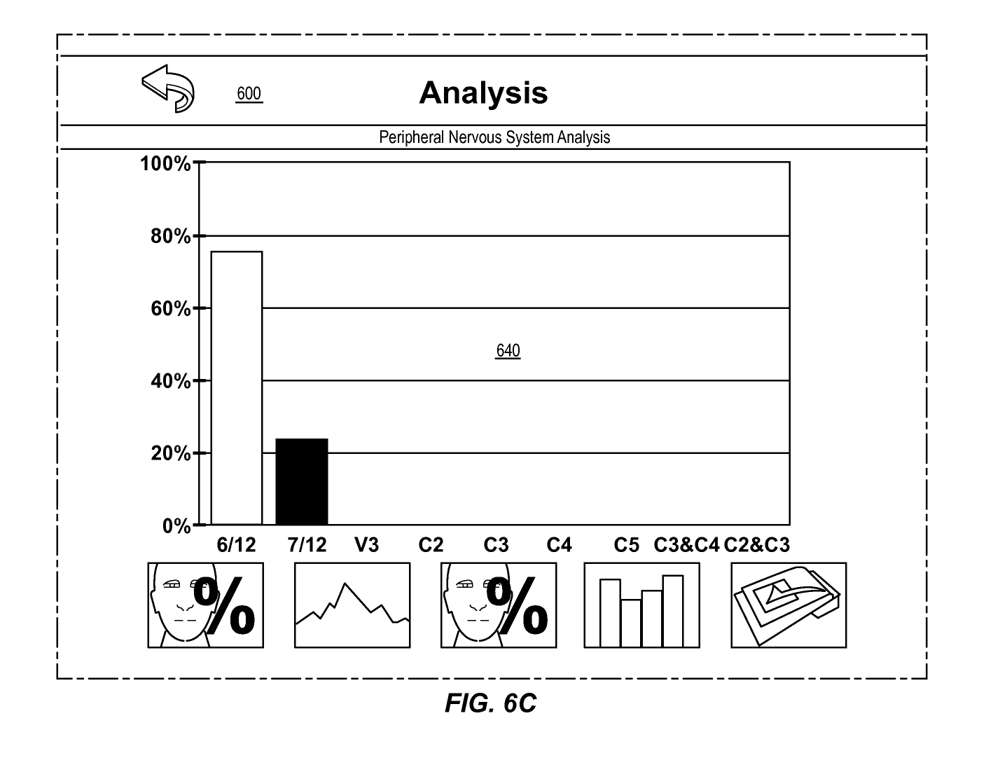

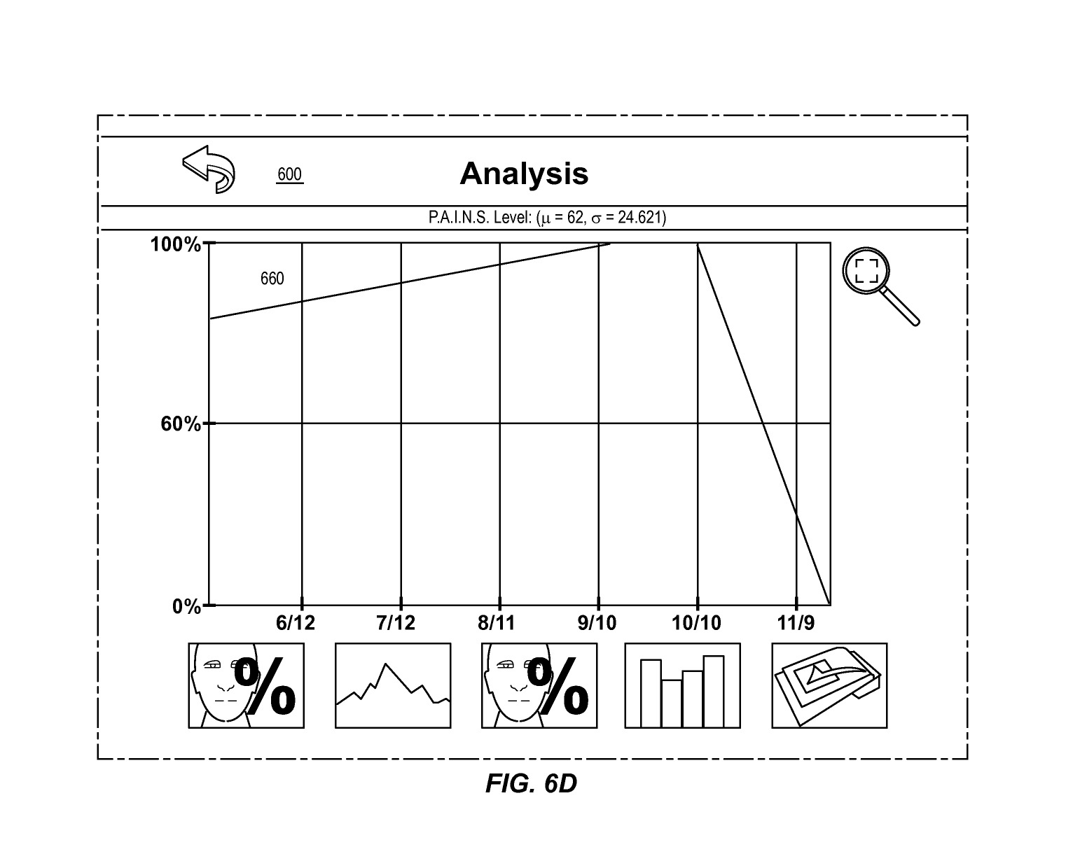

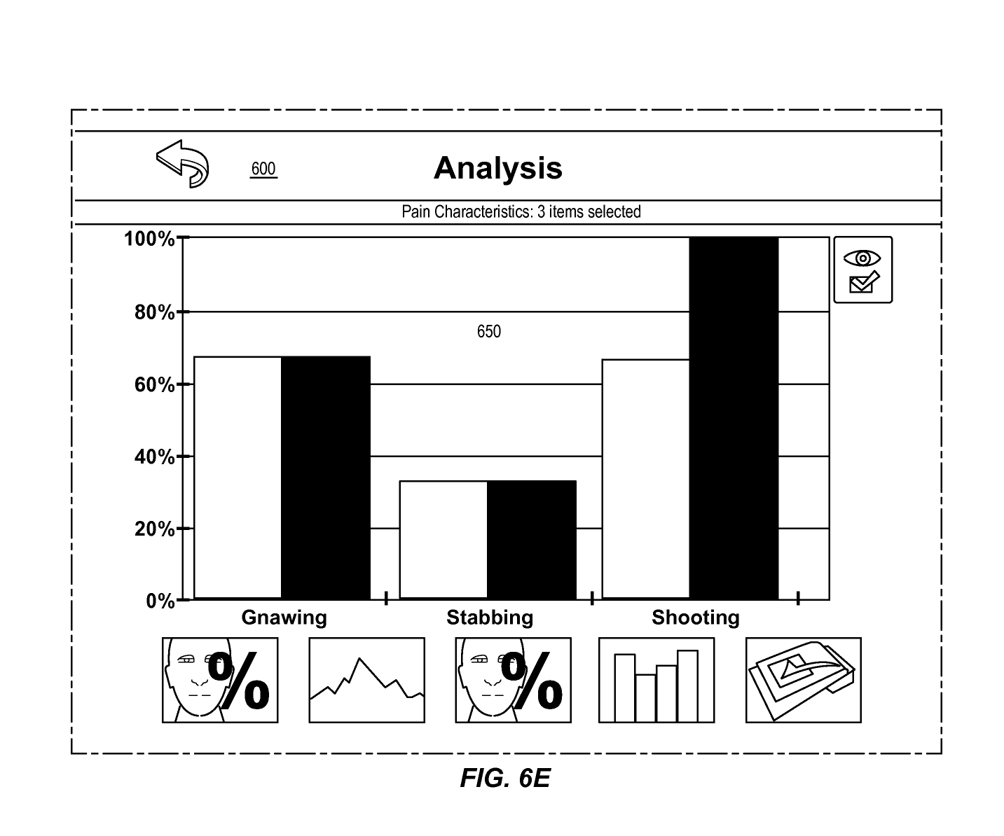

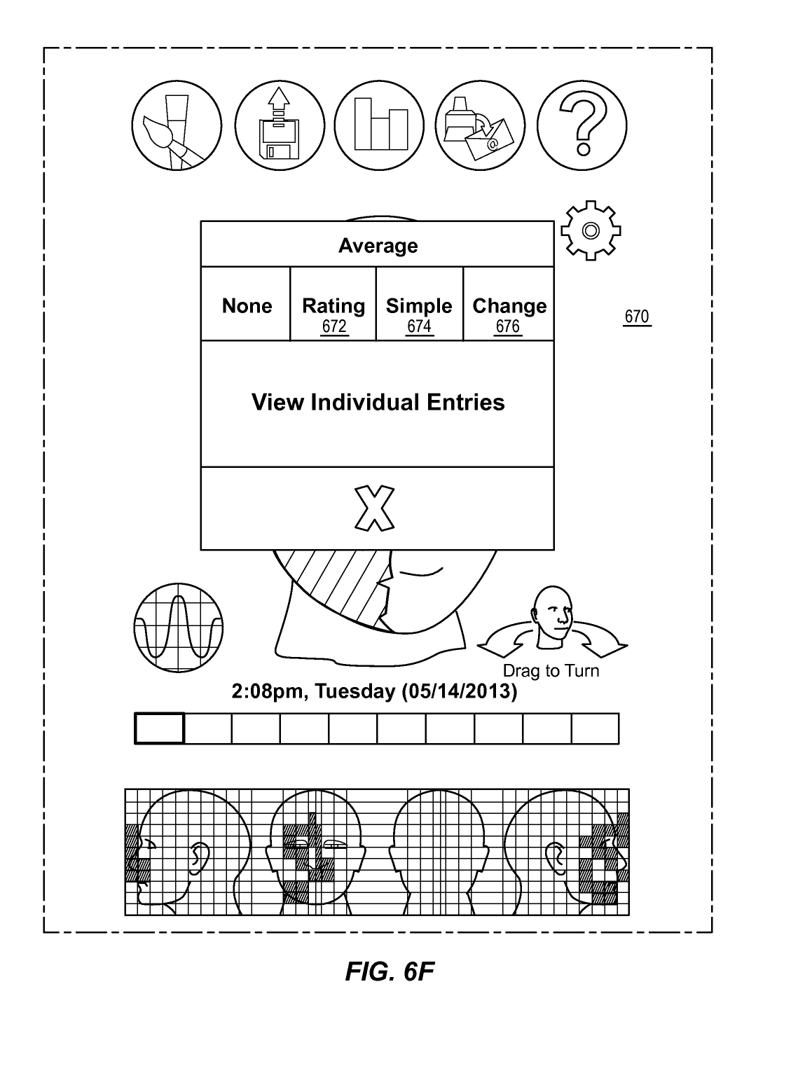

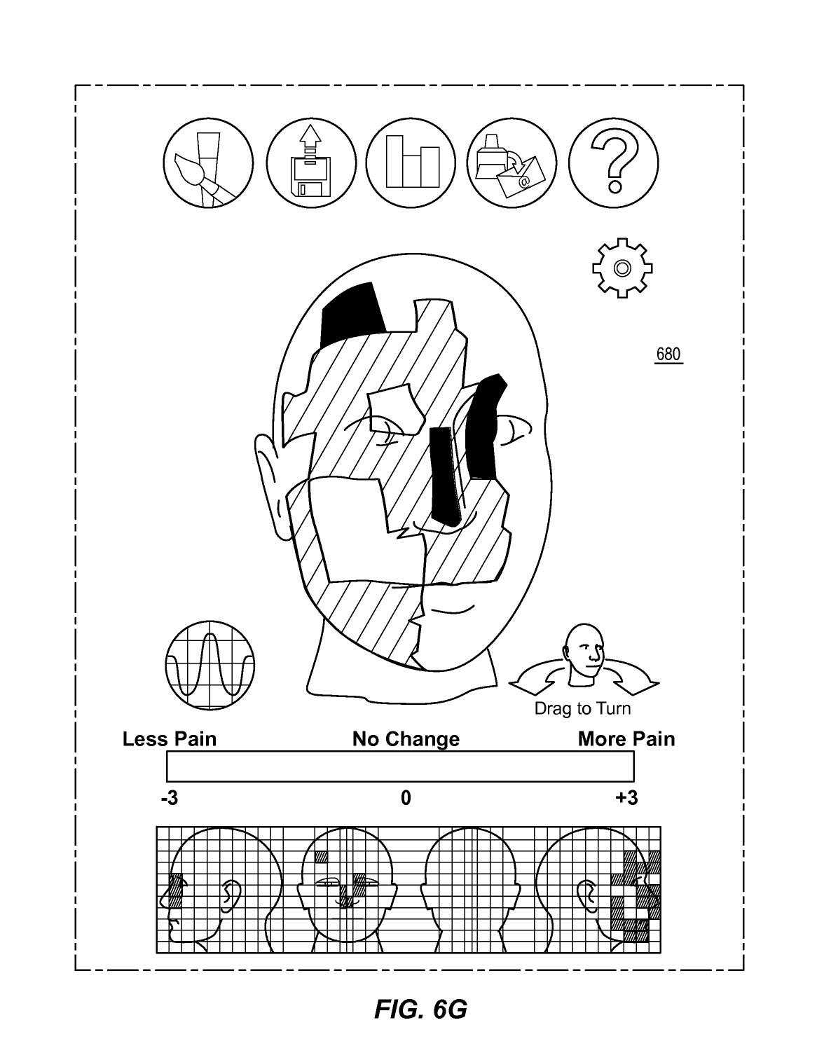

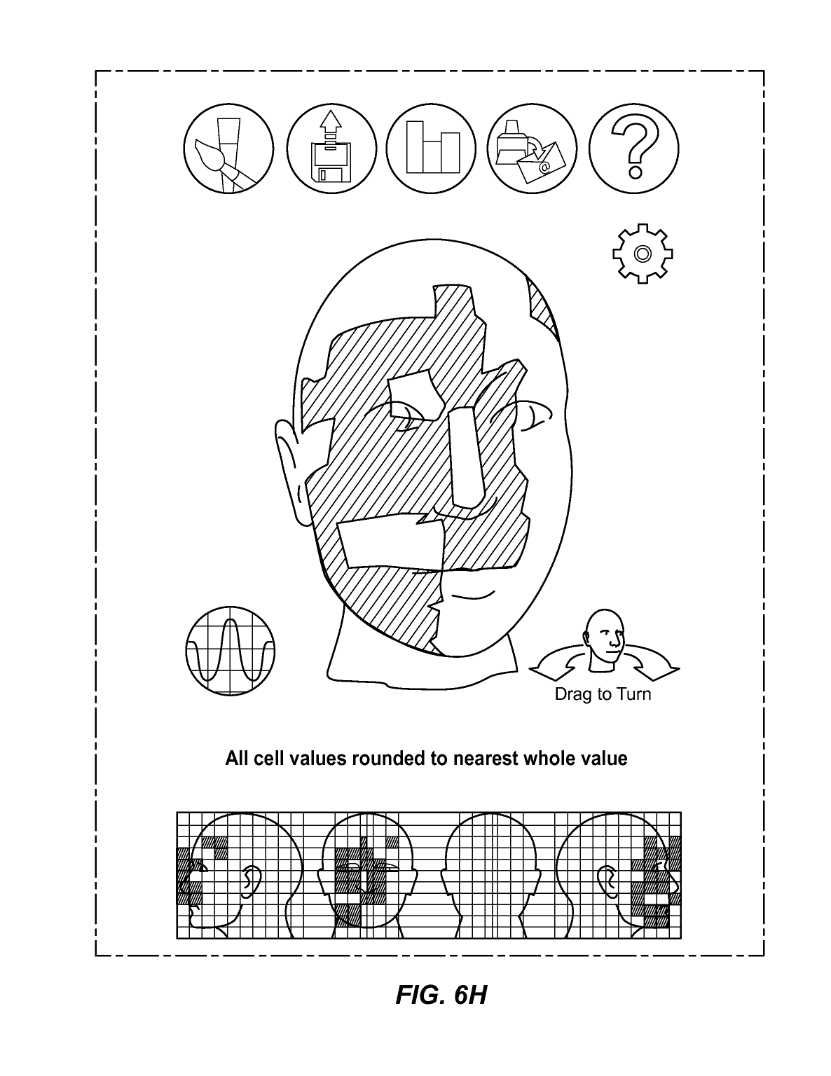

Referring again to FIG. 5B, loaded pain data screen 520 also displays menu buttons including clear currently loaded pain data button 530, save current pain data button 532, data visualization button 534, send data button 536, help button 538, settings button 540, and data averaging method selection button 542. Selecting data visualization button 534 causes display of data visualization screen 600 as shown in FIG. 6A. Data visualization screen 600 contains data analysis display area 602 and data analysis selection buttons 604, 606, 608, 610, 612. As with all data visualizations described herein, the pain data sets shown in the visualization are the pain data sets selected by the user in load screen 500. FIG. 6A is a plot 614 showing average pain level over the course of the loaded pain data sets. Plot 614 indicates mean 616 and standard deviation 618 of the loaded data set. Average pain level plot 614 is selected on data visualization screen 600 via average pain button 604. A user may manipulate average pain level plot 614 via zoom control 620.

FIG. 6B is a screen shot of data visualization screen 600 displaying average pain area plot 630, which is selected via average pain area button 606. Average pain area plot 630 also indicates mean pain coverage and standard deviation of the pain coverage. FIG. 6C illustrates data visualization screen 600 displaying a peripheral nervous system bar graph 640, accessed via peripheral nervous system button 610. Peripheral nervous system bar graph displays dermatome affected by pain locations in the loaded data set shown in load screen 500. Similarly, FIGS. 6E and 6D shows pain characteristics bar graph 650 and P.A.I.N.S. level plot 660 on data visualization screen 600, accessed via buttons 612 and 614, respectively. The values of P.A.I.N.S. level plot 660 may be a raw number or a percentage specific to a region of the body, e.g., head, upper body, or full body.

Returning now to FIG. 5B, loaded pain data screen 520, the device may display aggregate data visualizations directly on 3D head figure 202 according to any of several available methods: a rating average, a simple average, or a change in pain level. As with the information displayed on data visualization screen 600, any aggregate data displayed on 3D head figure 202 is drawn from the pain data sets selected by the user on pain load screen 500. The user may select 3D presentation method button 542 to display 3D presentation method screen 670, illustrated as FIG. 6F. 3D presentation method screen 670 displays rating average button 672, simple average button 674, and change in pain button 676. When selected, each choice leads to a 3D head figure visualization screen. For example, change in pain button 676 displays pain change screen 680 as shown in FIG. 6G; simple average button 674 causes display of FIG. 6H; and rating average button 672 causes display of FIG. 6I.

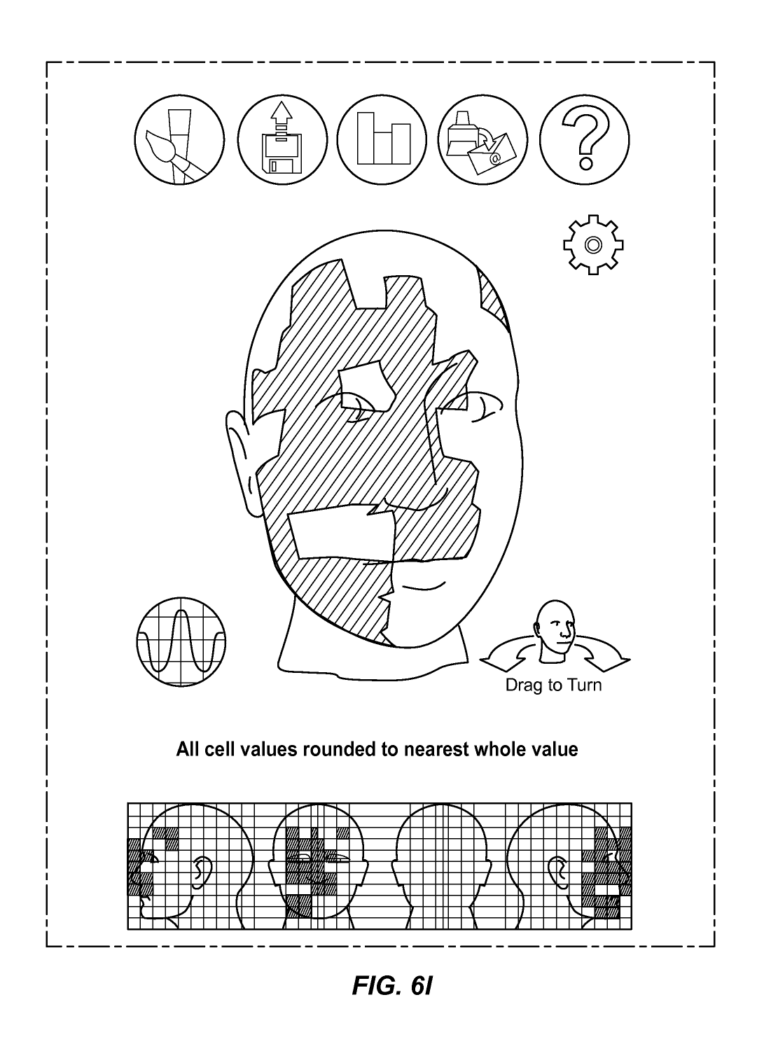

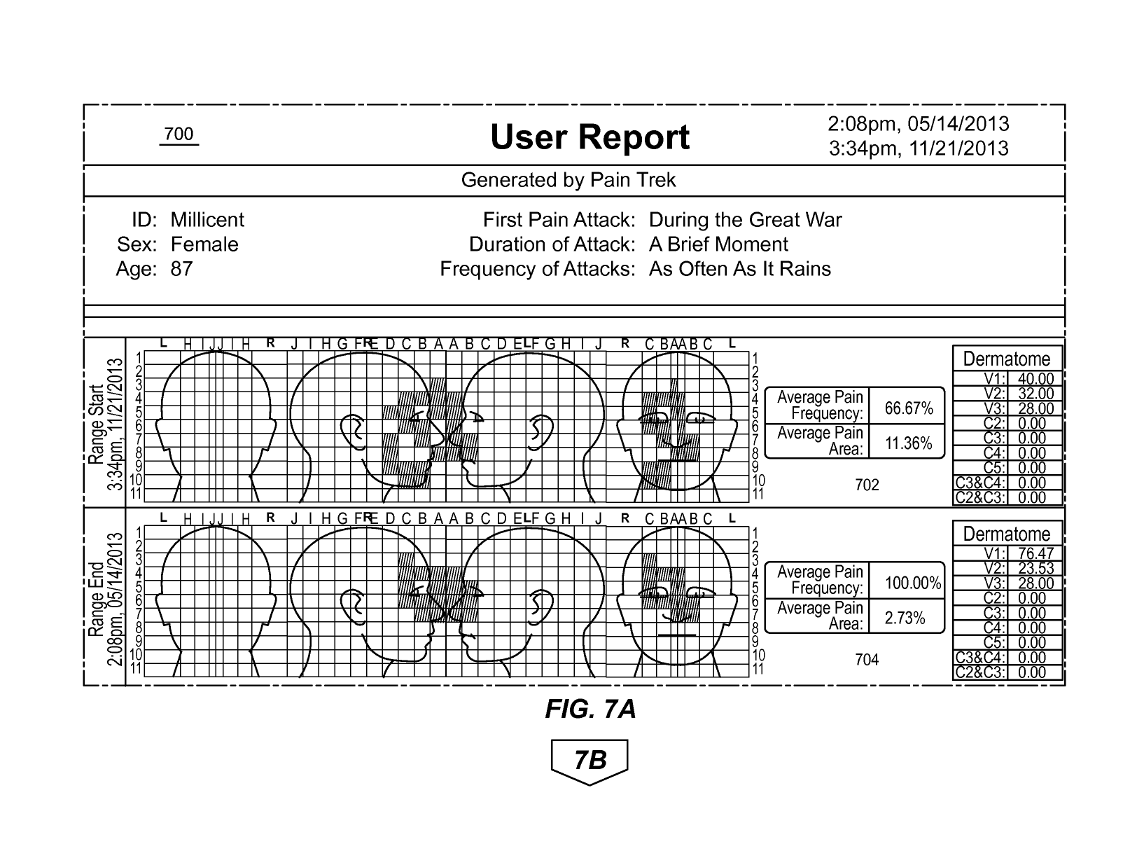

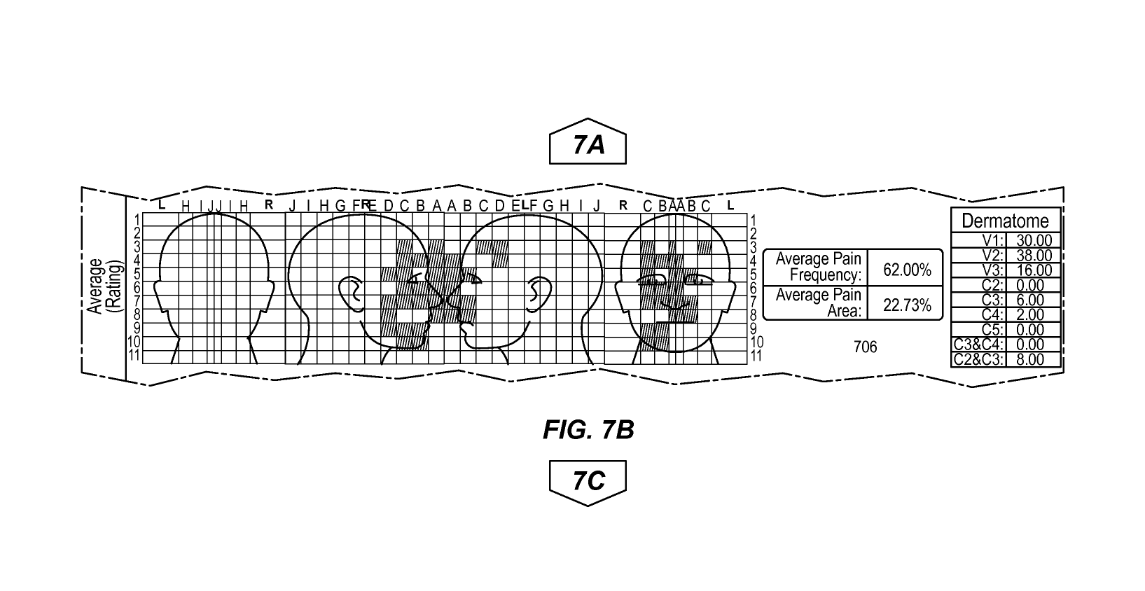

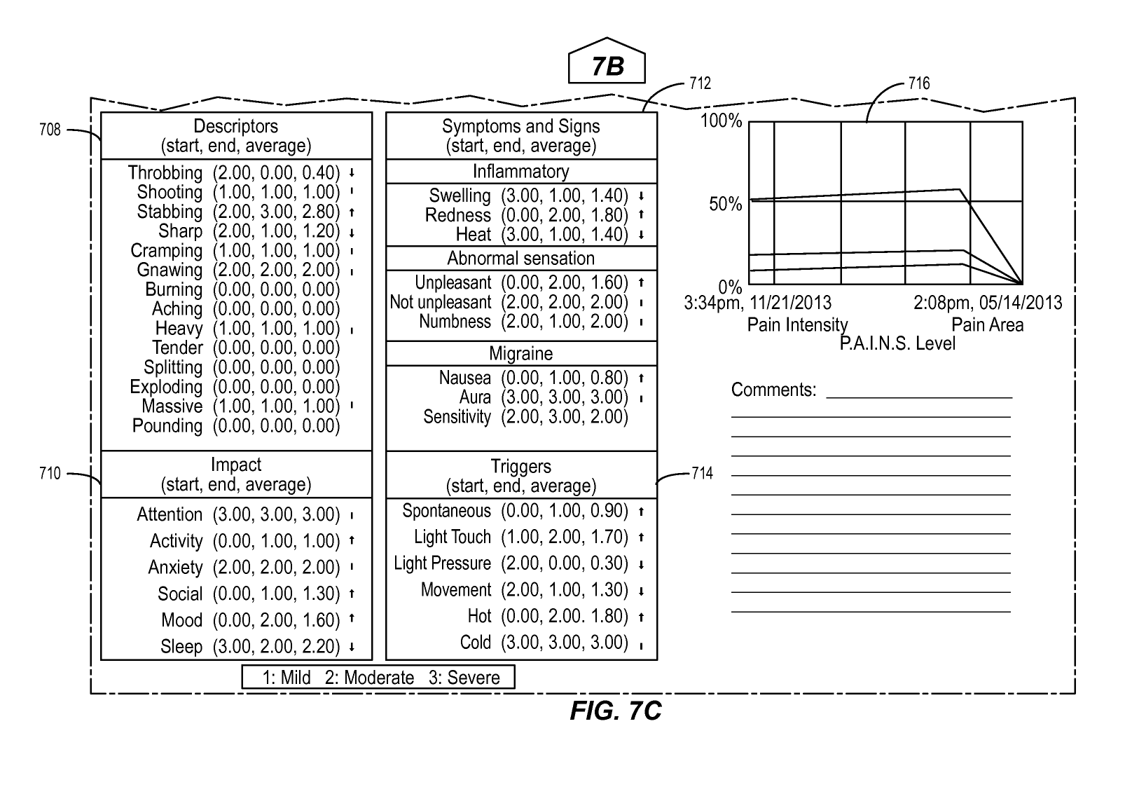

Data collected according to the method described above may be summarized in a user report such as user report 700 illustrated in FIGS. 7A-7C. User report 700 displays pain data on anatomical grids 702, 704, 706, as well as descriptive pain data in table 708, impact data in table 710, symptoms and signs data in table 712, and trigger data in table 714. User report 700 may further display one or more pain plots such as pain intensity, pain area, etc in plot area 716.

FIGS. 8A and 8B illustrate data schema for a patient's user data and a patient's input pain data, respectively. The rows in FIG. 8A indicate entries by date with indications of the levels of qualitative pain ratings entered by the patient on the respective dates. The rows of FIG. 8B indicate the rated pain levels for each of the sub-regions of the mapped bodily region.

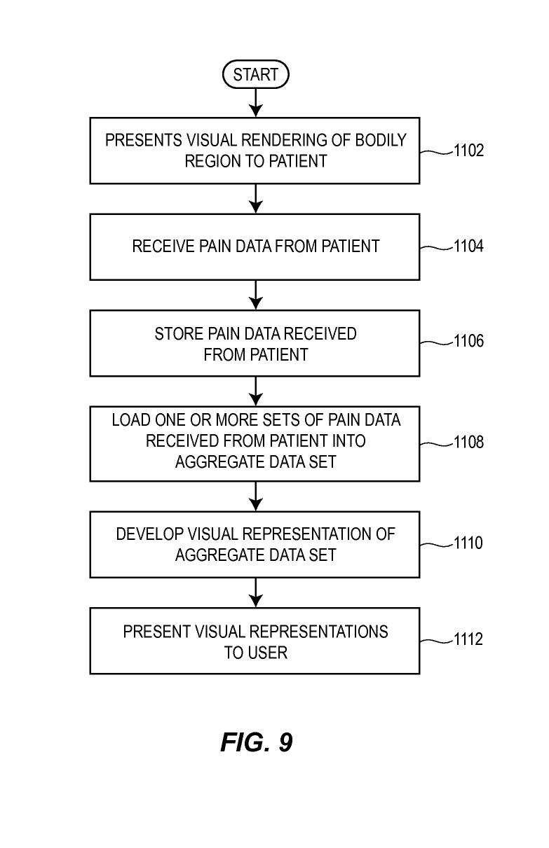

FIG. 9 is a flow diagram of a patient pain data collection, tracking, and analysis process 1100 that may be implemented by the system 100. Initially, a visual rendering of a bodily region is presented to the patient at block 1102 either in the form of a 3D bodily rendering or one or more anatomical grid interfaces. The system, at block 1104, may receive pain data, including pain location, pain intensity, and qualitative pain data, from the patient at block 1102 according to the user interfaces described above. At block 1106 the system 100 may store the received pain data in one or more memories such as memory interfaces 140 or 150 as described above. At block 1108 the system 100 may load one or more saved pain data sets according to load data screen 500 as described above into a single aggregate set of patient pain data. At block 1110, the system 100 may develop visual representations of the aggregate data set as described above including: average pain level, average pain coverage, rated pain average, implicated dermatome areas, pain gain or loss, P.A.I.N.S. level, collected pain characteristics, reported impact, reported symptoms, reported triggers, among others. At block 1112, the system 100 may present the developed visual representation of the aggregate data set to the user.

Experiment 1

The aggregation techniques were applied in an example experiment to assess pain onset. In particular the techniques were used to evaluate, in vivo, the μ-opioid system during spontaneous episodic migraine headaches. Patients were scanned at different phases of their migraine using Positron Emission Tomography (PET) with the selective μ-opioid receptor (μOR) radiotracer [11C] carfentanil. We determined that, in the ictal phase, there was μOR activation in the medial prefrontal cortex, which was strongly associated with the μOR availability level during the interictal phase. Furthermore, μ-opioid binding changes showed moderate negative correlation with the combined extension and severity of the attacks. These results indicated for the first time that there is high μOR activation in the migraineurs' brains during headache attacks in response to their pain.

Patients with chronic migraines routinely use opioids for treatment. Although the endogenous opioid system has long been implicated in regulating pain nociceptive signals, frequent use of opioids increases the risk of chronification of the migraine attacks and even allodynia. Hence, the status quo of the endogenous μ-opioid release and μOR concentrations during headaches are useful elements for the understanding of the neurobiology of migraine and, most importantly, its clinical alleviation or aggravation.

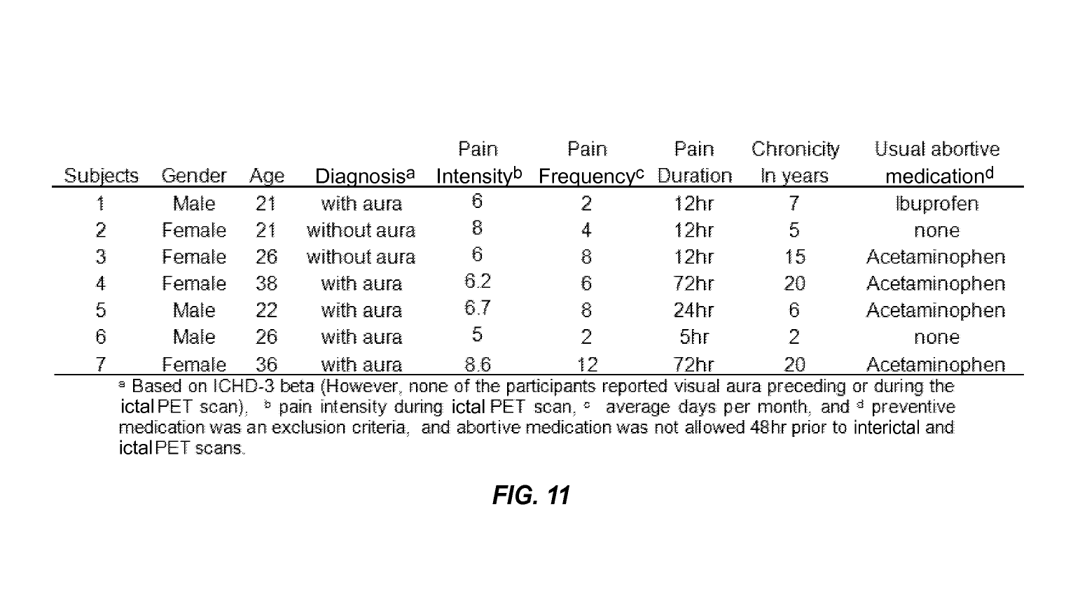

The experimental protocol was as follows. After initial screening by telephone, patients were thoroughly examined by a pain specialist to confirm the episodic migraine diagnosis following the International Headache Society classification (see, FIG. 11). Subjects were excluded in cases of opioid and hormonal contraceptive use during the past six months, pregnancy, and concomitant chronic pain conditions. The protocol was divided into one screening appointment, one MRI session, and two PET sessions: one during headache (ictal) and another during non-headache (interictal) phases of their migraine. Interictal phase also required participants to be headache free for at least 48 hours prior to the scan, and to have abstained from the use of any migraine medication during the same period. Both PET scans were scheduled a priori, and the patients had to confirm in the early morning of those days the occurrence, or not, of the attacks. For females, the PET sessions were arranged during separate mid-late follicular phases (5-10 days after menstrual bleeding) with the assistance of a gynecologist.

Ictal and Interictal PET sessions: PET sessions with [11C]carfentanil (CFN), a selective and specific μ-opioid receptor radioligand, were performed for 90 minutes. PET scans were acquired with a Siemens HR+ scanner in 3-D mode (reconstructed FWHM resolution [symbol] 5.5 mm in-plane and 5.0 mm axially) with septa retracted and scatter correction. Subjects were positioned in the PET scanner gantry and two intravenous (antecubital) lines were placed. [11C]carfentanil was produced using a cyclotron in the vicinity, and each dose (15±1 mCi, <0.03 μg/kg) was administered fifty percent as a bolus with the remainder continuously infused over the course of the scan to achieve steady-state tracer levels approximately 35 minutes after tracer administration.

Electronic mobile pain data entry: Headache and facial pain intensity and area data were collected and analyzed using the pain tacking application, such as the pain analysis module described above. Patients identified regions of pain on the 3D rendering of the head to express their exact migraine headache location and intensity, as well as other pain characteristics. The pain tracking application automatically calculated and displayed the rating of average pain intensity and extension for all patients together. This determination included the total sum of patient(s)' pain severity in each anatomical location, divided by the number of responses in the area (Mild:1/Moderate:2/Severe:3). Anatomical regions without pain were considered null responses and not counted in the rating average. Also, the application accounted for the overall pain for each participant by determining the Pain Area and Intensity Number Summation (P.A.I.N.S) of all rated regions of the 3D rendering (i.e., the polygons/squares) together. This approach showed the precise anatomical distribution and intensity of the migraine attacks studied across all our patients or individually, providing a more objective and detailed sensory-discriminative information of the attacks.

MRI Acquisition: MRI scans were acquired on a 3T scanner (General Electric, Milwaukee, Wis.). These images provide anatomical information for structure identification and were utilized for the anatomical standardization to the ICBM/MNI atlas coordinate system. This established the linear and non-linear warping transformation matrices applied to the co-registered receptor binding PET maps. The acquisition sequence was axial T1 FAST SPGR MR (TE=3.4, TR=10.5, TI=200, flip angle 25 deg, FOV 24 cm, 1.5 mm thick slices, NEX=1), acquisition matrix 256×256, 60 slices.

Neuroimaging Analysis:

T1-weighted MRI and PET images of each subject were co-registered to each other using a mutual information algorithm. For this purpose, K₁ ratio images were first aligned to the MRI, and the transformation matrix applied to the co-registered BPND scans of the same image set. The MRI scans were then anatomically standardized to ICBM brain atlas stereotactic coordinates by non-linear warping, and the resulting transformation matrix applied to both K₁ ratio and BP_{ND} image sets.

Subsequently, dynamic image data for each of the receptor scans were transformed on a voxel-by-voxel basis into two sets of parametric maps, which were co-registered to each other. These were a tracer transport measure (K₁ ratio, proportional to cerebral blood flow; tracer transport=blood flow×tracer extraction) and receptor-related measures (non-displaceable binding potential, BPND), encompassing data from 10-40 min (baselines). These parametric images were calculated using a modified Logan graphical analysis with the occipital cortex (a region devoid of μ-opioid receptors) as the reference region.

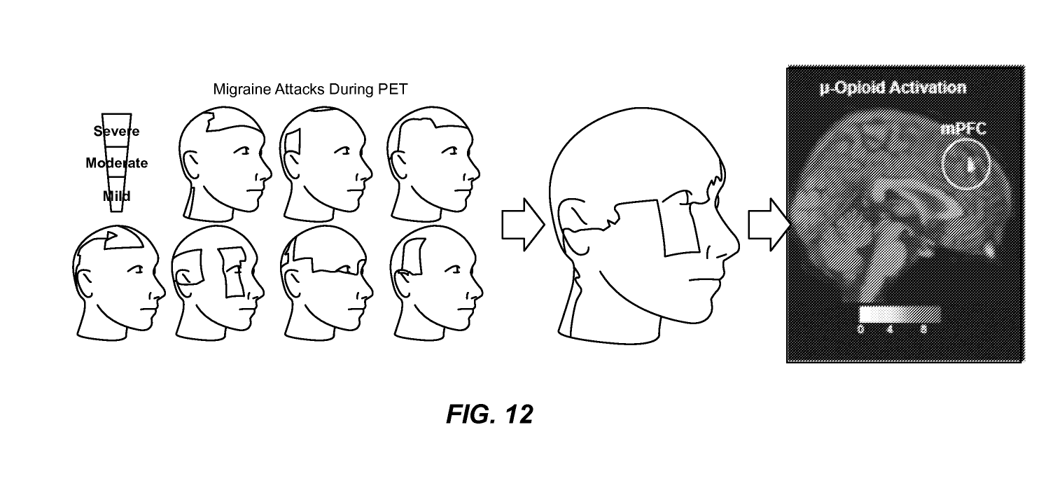

Of the twelve episodic migraine patients scanned during their interictal phase, seven patients (four females/three males) confirmed by phone, upon awakening, the occurrence of their spontaneous migraine when scheduled a priori for their potential ictal PET scans. Clinical characteristics of the migraine headache are summarized in table 1. Participants managed to tolerate the headache attacks until the end of the scan sessions without any abortive pharmacotherapy. The average intensity of the headache attacks was moderate (6.6±1.6 (VAS (1-10)) and pain extension was 39±26.7 square units (FIG. 12, center image). With the exception of patient 1, all other patients had migraine predominantly on the right side. For clinical and neuroimaging analysis, patient 1's data was flipped. No additional migraine attacks were reported by the patients during the three days that preceding or following the ictal phase scanned. Their average frequency of attacks was 6±3.6 per month, and history of 11.1±7.1 years of migraine suffering.

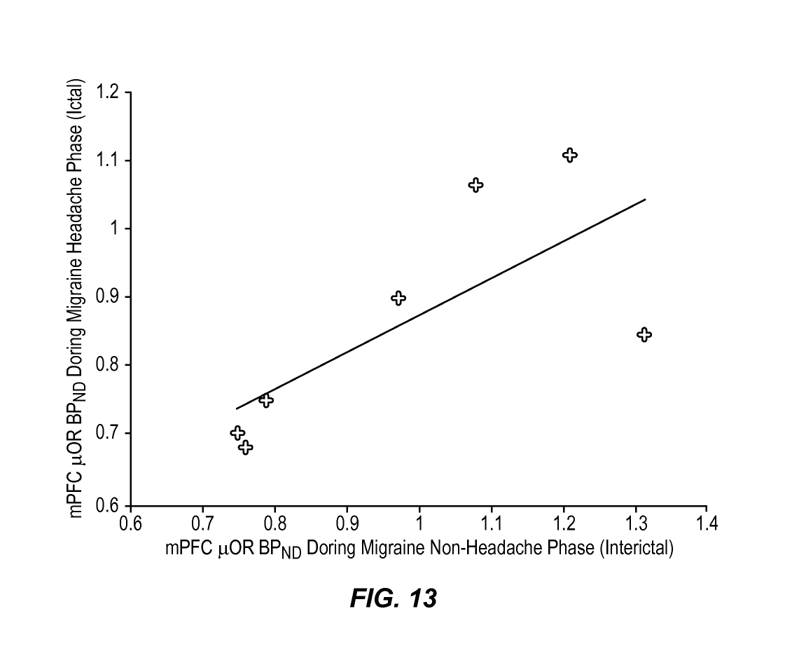

We found reductions in μOR BPND during a spontaneous migraine attack compared to the baseline in the medial Prefrontal Cortex (mPFC) ipsilateral to the headache (MNI coordinates with a center of mass at right: x: 2; y: 43; z: 42; p=0.000) (FIG. 12 rightmost side). These results indicated the acute activation of the endogenous opioid neurotransmission interacting with μOR due to the pain of the migraine attack. The μOR BPND in the mPFC cluster during the ictal migraine phase was positively correlated with the μOR BPND levels during interictal phase (r:0.74) (FIG. 13). No correlations were found with the averages of attack intensity, extension or frequency separately. However, when intensity and extension of the current headache attacks were accounted for together (P.A.I.N.S) there was a moderate negative correlation with mPFC activation (r:−0.61).

Thus, by collecting and analyzing pain information from patient interaction with the 3D rendering display we were able to demonstrate, in vivo, that there was reduced μOR BPND in the central modulatory pain system of migraine patients during spontaneous headache, compared to their non-headache phase. There were less μ-opioid receptors available for binding for the specific PET radiotracer [11C] Carfentanil in the ipsilateral mPFC during the ictal phase, possibly due to the increased endogenous μ-opioid neurotransmission interacting with μORs. This implies that the migraine headache attack induced the release of the endogenous μ-opioids to fight the ongoing pain. However, due to the continuation of the migraine throughout the scan it can be inferred that the higher endogenous μ-opioid activation was ineffective to control the barrage of nociceptive inputs associated with the migraine headache pain. The continuation of pain along with the decreased BPND of the [11C]carfentanil during the ictal phase in the mPFC as compared to the interictal headache phase show an association between endogenous μ-opioid release and migraine headache pain in this area of the brain.

The mPFC region, including the rostral anterior cingulate cortex, had been linked, although indirectly, to migraine attacks by other animal and human studies. This region processes the cognitive-emotional and spatio-temporal variables associated with spontaneous clinical pain. The μOR activation of that region increases connectivity with the periaqueductal gray matter (PAG) in analgesia, another region rich in μOR and involved in migraine pathophysiology. With a migraine, functional activation in the prefrontal region has been previously noticed in spontaneous and triggered migraine attacks. In addition, meningeal neurogenic inflammation associated with migraine can be modulated in animal studies by morphine, and afterward, overturned by naloxone, a μ-opioid antagonist. Nevertheless, based on our preliminary findings, the imbalance between the faulty descending inhibition and the facilitation of the ascending trigeminal sensory inputs must both be present during the occurrence of the migraine symptomatology. Otherwise, only the acute increase in the release of endogenous μ-opioid we observed at the time of the attacks would be enough to cease the patients' suffering, which was not the case. Furthermore, we observe that the level of this μ-opioid activation fluctuates depending on the migraine experience, as it weakens with the progression of the area and severity of the migraine attack, showing a moderate negative correlation with the pain summation (P.A.I.N.S).

Experiment 2

The use of opioids in clinical practice is not without risk of undesired effects, especially in migraine patients where the recurrent nature of the attacks, and consequently the frequent use of rescue opioid intake, can severely increase the risk of chronification and even allodynia. This augmented cutaneous sensitivity to stimuli that should not cause pain, already present in 65% of migraineurs, turns mundane activities such as washing the face with hot water and combing the hair into distressing tasks during the headache attacks. In Experiment 1, we demonstrated that there was an ineffective high release of endogenous μ-opioids at the cortical level to fight the ongoing migraine pain. More precisely, this was noted in the medial prefrontal cortex (mPFC), a cortical area that processes the spatio-temporal and cognitive-emotional inputs related to spontaneous chronic clinical pain.

In this experiment, Experiment 2, we seek further information regarding the involvement of the endogenous μOR system in the allodynic response during migraine attack. Such information could provide a molecular explanation of why certain patients have increased cutaneous sensitivity. As with Experiment 1, we use the increased accuracy of the paint tracking and analysis techniques described herein to collect accurate pain information that facilitates measurement and assessment of brain activity in migraine formation and subsequent treatment.

For Experiment 2, in order to address the technical requirements for molecular neuroimaging in humans we used a sustained thermal pain threshold (STPT) challenge, that we developed, on the trigeminal ophthalmic region. With this, we were able to examine for the first time in vivo, changes in μOR activity in the brains of migraine patients during the ictal allodynic experience.

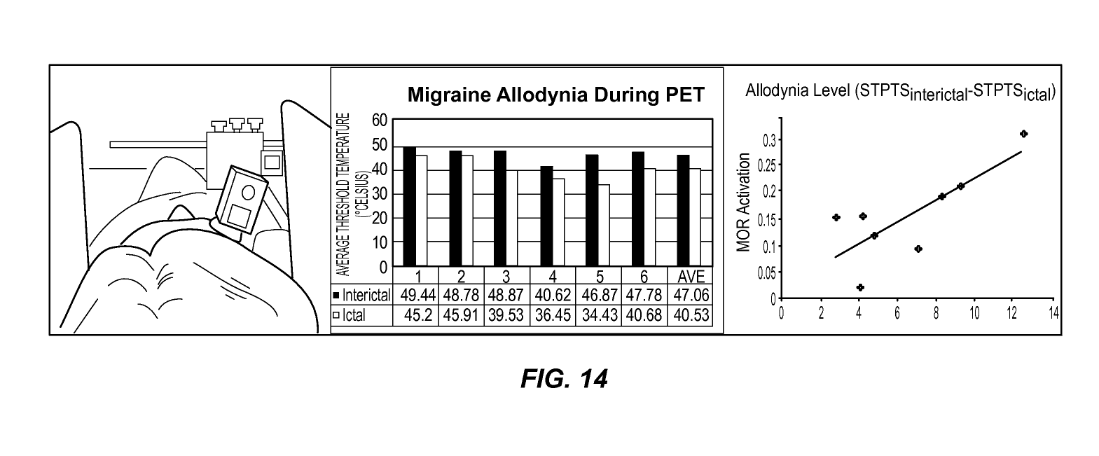

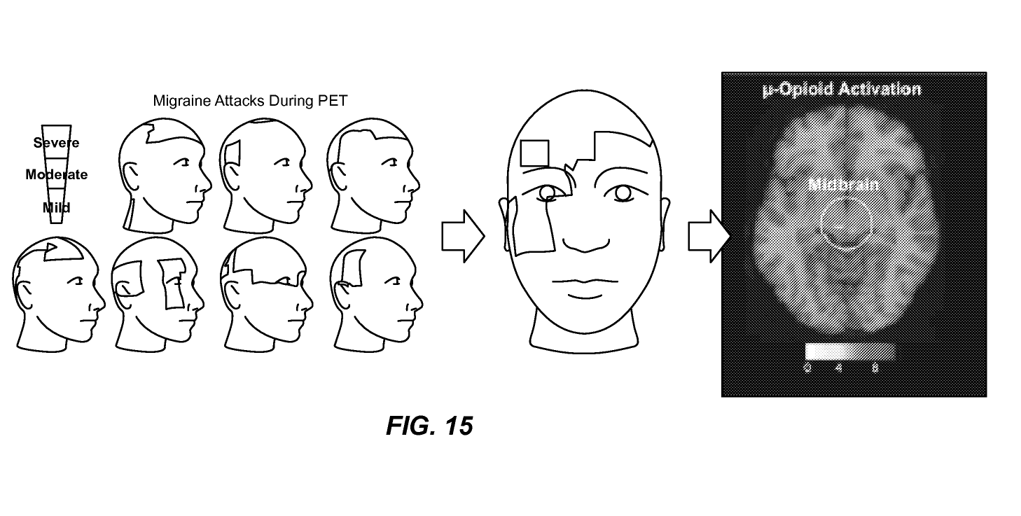

Sustained Thermal Pain Threshold (STPT)—PET Challenge: the STPT in the trigeminal ophthalmic region was developed in-house for various reasons, including technical elements related to receptor quantification PET methods (FIG. 14). Receptor binding measures in PET require the utilization of challenges sufficiently long in duration so that a constant state can be achieved and enough data points collected to permit quantification. The heat intensity was controlled by the individual's experience, from a starting baseline of 32° C., multiple heat cycles occurred at constant rates (1° C./Sec ascending and descending), and applied to the forehead area (V1) ipsilateral to the headache using a 16 mm² thermal probe system (Pathway Model—MEDOC, Israel). The subjects were instructed to tap the mouse button at the first perception of pain to instantly return temperature to baseline level. In that manner, individuals with migraine selected their thermal pain threshold based on their current sensitivity, which avoided unnecessary discomfort during the experiment, especially in the allodynic ictal sessions. The challenge cycles were repeated every 10 sec for 20 min during the PET session, and multiple pain thresholds measurements were recorded to provide the average threshold of the session (FIG. 15—leftmost side).

Seven patients (four females/three males) contacted us by phone in the early morning with spontaneous migraine for their ictal PET scans. They were instructed to tolerate the pain without any rescue pharmacotherapy until the end of the scan sessions. The seventh patient's allodynia phase data was eliminated due to thermal probe displacement during scan. The average pain intensity of the remaining patients was moderate (6.3±0.9 VAS (1-10)) for the headache attacks. With the exception of patient 1, all other patients had migraine predominantly in the right side (FIG. 11). All the patients showed significant cutaneous heat allodynia during the ictal PET session in the ipsilateral ophthalmic trigeminal area when compared to the interictal phases (p<0.003) (FIG. 15—Center). No additional headache attacks were recounted by the patients during the three days before or after the ictal phase scanned.

We also noticed a decrease in μOR BPND during the cutaneous heat allodynia associated with the spontaneous migraine attack. There were concurrent bilateral clusters of endogenous μOR activation in the midbrain, extending from the red nucleus (RN) to the ventrolateral periaqueductal gray matter (vlPAG) (MNI coordinates with a peak on the left side: x: −6; y: −20; z: −8; p<0.000) (FIG. 15), which was positively correlated with the patients's allodynic levels (p<0.003; r: 0.75) (FIG. 15—Right). These results indicate the acute activation of endogenous opioid neurotransmission interacting with μOR due to the allodynic experience of the migraine attack.

Thus Experiment 2 demonstrated for the first time in vivo demonstration of the μ-opioid system involvement in cutaneous migraine allodynia during spontaneous attacks. Increased endogenous μ-opioid neurotransmission interacted with μORs particularly in the vlPAG and red nucleus, important midbrain areas related to migraine pathophysiology and allodynia modulation. Moreover, these flawed μOR activations were positively correlated with the severity of the patients' trigeminal allodynia. These findings indicate that, in addition to the migraine headache attack, the abnormal allodynic cutaneous experience was concurrent with ineffective high-release of endogenous μ-opioids.

The PAG is a crucial supraspinal site of the antinociceptive descending pathway that also includes the rostral ventromedial medulla (RVM) and the dorsal horn of the spinal cord. The RN participates in cognitive circuits related to salience and executive control, as well as in the modulation of allodynia. In migraine patients, there is a significant increase of iron deposition in both regions, which positively correlates with the duration of the illness. Our experiments confirm that there is increased endogenous μ-opioid neurotransmission interacting with μORs accompanying the intensification of the trigeminal allodynic experience and the migraine suffering.

μOR BPND is a measurement in vivo of endogenous μ-opioid receptor availability, and its instant decrease reflects the triggering of this neurotransmitter system during allodynic migraine suffering. The same cohort of migraine patients was previously used to report reduced μOR BPND in the medial prefrontal cortex (mPFC) solely during the headache phase before the thermal challenge, which was found to be negatively correlated with the combined measure of pain area and intensity (Pain Area and Intensity Number Summation—P.A.I.N.S) (DaSilva A F et al. “Association of μ-Opioid Activation in the Prefrontal Cortex with Spontaneous Migraine Attacks—Preliminary Report I”. Submitted, 2013). It is known that μOR activation of the mPFC increases connectivity with the PAG in analgesia 19.

Remarkably, we found a key difference regarding the level of μ-opioid release in mPFC regions when a brief migraine allodynic experience takes place. Although μ-opioid release weakened with the extension and severity of the migraine pain in Experiment 1, the system showed the opposite behavior with the focal allodynic experience. This was demonstrated in the current study by the positive correlation we found between μ-opioid release in the vlPAG cluster with the ictal allodynic severity.

It is possible that the salient and dysfunctional cutaneous sensory experience during our migraine protocol triggers further activation of the central μ-opioid system to respond to a potential external threat and ongoing pain, possibly represented by the additional ascending trigeminal sensory inputs. This explains the partial ineffectiveness of anti-migraine medication once central sensitization with cutaneous allodynia is established in the late phase of headache attack, since there is already a concurrent overflow of endogenous μ-opioids acting on the existent μOR20. Despite targeting one of the more important analgesic receptor-based mechanisms in the brain, these drugs are competing with the patients' own endogenous pain relieving systems. In fact, the prior use of opioids alters treatment resistance to even non-opioid analgesic drugs in migraine patients20. Hence, opioids are not recommended as the first choice for the treatment of migraine by the US Headache Consortium Guidelines, and it should be reinforced that their use in clinical practice is not evidence based.

In conclusion, we found additional release of endogenous μ-opioids acting on μOR during cutaneous migraine allodynia in the midbrain region, including the vlPAG and RN, which was positively correlated with the ictal changes in skin sensitivity to heat pain. Further studies should be conducted to evaluate how this endogenous μ-opioid mechanism is related to allodynia in other pain disorders and migraine subtypes, including chronic migraine. These novel results in vivo oppose the common practice of using opioids as rescue therapy for episodic migraine patients, especially for those with established allodynia, as there is already high central occupancy of μ-opioid receptors.

It will be appreciated that the above descriptions are provided by way of example and that numerous modifications may be made within context of the present techniques.

More generally, the various blocks, operations, and techniques described above may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any computer readable memory such as on a magnetic disk, an optical disk, or other storage medium, in a RAM or ROM or flash memory of a computer, processor, hard disk drive, optical disk drive, tape drive, etc. Likewise, the software may be delivered to a user or a system via any known or desired delivery method including, for example, on a computer readable disk or other transportable computer storage mechanism or via communication media. Communication media typically embodies computer readable instructions, data structures, program modules or other data in a modulated data signal such as a carrier wave or other transport mechanism. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Thus, the software may be delivered to a user or a system via a communication channel such as a telephone line, a DSL line, a cable television line, a wireless communication channel, the Internet, etc. (which are viewed as being the same as or interchangeable with providing such software via a transportable storage medium).

Moreover, while the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

Thus, although certain apparatus constructed in accordance with the teachings of the invention have been described herein, the scope of coverage of this patent is not limited thereto. On the contrary, this patent covers all embodiments of the teachings of the invention fairly falling within the scope of the appended claims either literally or under the doctrine of equivalents.

What is claimed:

1. A method of tracking and analyzing pain experienced by a subject, the method comprising:
presenting, on a display, a visual rendering of a bodily region to track and analyze pain, where the visual rendering comprises a plurality of sub-regions collectively mapping the bodily region, where each sub-region is individually selectable by the subject;
receiving, from the subject interacting with the visual rendering on the display, identified pain data to create one or more pain heat maps, where each heat map comprises (i) a selection of one or more of the sub-regions and (ii) an indication of pain intensity for each of the selected one or more sub-regions, where the indication of pain intensity is a numeric value taken from a pain intensity scale;
developing, from the one or more pain heat maps, an aggregated pain data set for the bodily region, the aggregated pain data set including averaging data indicating an average pain intensity value over the one or more pain heat maps, sub-region coverage data indicating a percentage of plurality of sub-regions selected by the subject over the one or more pain heat maps, and summation data indicating a sum of total pain intensity from the one or more pain heat maps; and displaying a visual representation of the aggregated pain data set.

2. The method of claim 1, wherein displaying the visual representation of the aggregated pain data set comprises mapping the aggregated pain data set to an aggregate pain heat map on a second visual rendering of the bodily region.

3. The method of claim 1, the method further comprising:
receiving the identified pain data at different times over an analysis period to create a plurality of pain heat maps;
collecting, from a medical imaging modality, biologic activation event data for the analysis period;
correlating the aggregated pain data set to the biologic activation event data to determine if the biologic activation events coincide, precede, or succeed pain onset.

4. The method of claim 3, wherein the subject is a human and the bodily region is the head of the subject.

5. The method of claim 4, wherein the biological activation event is μ-Opioid receptor activation.

6. The method of claim 3 wherein the medical imaging modality is a positron emission tomography (PET) scanner, computed tomography (CT) scanner, magnetic resonance imaging (MRI) scanner, functional near infra-red spectroscopy (fNIRS), magnetoencephalography, MEG, or single-photon emission computed tomography (SPECT) scanner.

7. The method of claim 3, wherein developing the aggregated pain data set comprises averaging the indications of pain intensity over one or more pain heat maps.

8. The method of claim 3, wherein developing the aggregated pain data set comprises averaging the indications of pain intensity over one or more pain heat maps for a plurality of subjects.

9. The method of claim 3, wherein developing the aggregated pain data set comprises determining a rating of change of pain over the analysis period.

10. The method of claim 3, further comprising determining, from the one or more pain heat maps, an aggregated pain intensity score for the subject.

11. The method of claim 10, further comprising correlating the aggregated pain intensity score to the biologic activation events.

12. The method of claim 3, the method further comprising:
collecting the biologic activation event data over the analysis period in response to an external device applying a treatment to the bodily region; and
correlating the aggregated pain data set to the treatment to determine an effectiveness in reducing pain experience by the subject.

13. The method of claim 1, wherein presenting the visual rendering of the bodily region comprises;
rendering a 3D model of the bodily region and dividing the 3D model into a polygonal grid, where each polygon of the 3D model corresponds to one of the sub-regions.

14. The method of claim 13, wherein each polygon comprises vertical and horizontal coordinates.

15. The method of claim 1 further comprising tracking the aggregated pain data set over a plurality of dermatomes.

16. The method of claim 15, wherein each of sub-region corresponds to a different peripheral and central dermatome.

17. The method of claim 15, wherein a plurality of sub-regions collectively correspond to at one of the plurality of dermatomes.

18. The method of claim 1 further comprising allowing a user to select the one or more pain heat maps from a set of heat maps.

19. The method of claim 1, wherein the identified pain data comprises an amount of pain perceived by the subject, an amount of blurred vision perceived by a subject, an amount of sharpness of the pain perceived by the subject, numbness experienced by a subject, halos observed by a subject, dizziness experienced by a subject, vomiting experienced by a subject, or sweating experienced by a subject.

20. The method of claim 3, wherein the subject is a human and the bodily region is an internal bodily region, the entire external bodily frame of the subject, or a sub-region of the external bodily frame.

21. An apparatus having a processor and a computer readable medium that includes instructions that when executed by the processor cause the apparatus to:
present, to a subject experiencing pain, a first visual rendering of a bodily region wherein the visual rendering comprises a plurality of sub-regions collectively mapping the bodily region;
collect, from the subject experiencing pain, one or more pain data sets wherein each pain data set comprises pain intensity and pain location data corresponding to one or more of the plurality of sub-regions;
develop, in a memory, the one or more pain data sets to produce an aggregate pain data set; and
perform, in a pain analysis module, a data analysis of the aggregate pain data set to visualize the pain data for presentation on a second visual rendering of a bodily region.

22. The apparatus of claim 21 wherein the presentation of pain data on the second visual rendering of a bodily region comprises an average pain heat map over the plurality of sub-regions.

23. The apparatus of claim 21 wherein the presentation of pain data on the second visual rendering of a bodily region comprises an average pain heat map over the plurality of rated sub-regions.

24. The apparatus of claim 21 wherein the presentation of pain data on the second visual rendering of a bodily region comprises a heat map indicating change in pain intensity over the aggregate pain data set.

25. The apparatus of claim 21 wherein the first visual rendering of a bodily region and the second visual rendering of a bodily region comprise a 3-dimensional rendering.

26. The apparatus of claim 21 wherein the first visual rendering of a bodily region and the second visual rendering of a bodily region comprise a rendering of a human head.

27. The apparatus of claim 21 wherein the first visual rendering of a bodily region and the second visual rendering of a bodily region comprise an anatomical grid.

28. The apparatus of claim 21 wherein the data analysis further includes a user report.

29. The apparatus of claim 21 wherein the processor further causes the pain analysis module to export the aggregate pain data set.
